(19) United States
(12) Patent Application Publication
(10) Pub. No.: US 2026/0364353 A1
(43) Pub. Date: Nov. 19, 2026

(54) NEUROSTIMULATOR IMPLANT AND METHOD FOR TREATING SLEEP APNEA

(71) Applicant: Capri Medical Limited, Dublin (IE)

(72) Inventors: Fergal Ward, Dublin (IE); Ross Lewis, Dublin (IE); Ricardo Vallejo, Hallandale Beach, FL (US); Jorge Armando Cuartas, Antioquia (CO); Michael Herzog, Cottbus (DE); David Leonardo Cedeno, Parkland, FL (US); Willard Noyes, Bloomington, IL (US)

(73) Assignee: Capri Medical Limited, Dublin (IE)

(21) Appl. No.: 19/676,522

(22) Filed: May 13, 2026

Related U.S. Application Data
(60) Provisional application No. 63/805,708, filed on May 14, 2025.

(30) Foreign Application Priority Data
May 29, 2025 (IE) 2025/0231

(51) Int. Cl. A61N 1/36; A61B 5/00; A61B 5/08; A61N 1/05; A61N 1/372; A61N 1/375; A61N 1/378

(52) U.S. Cl. CPC A61N 1/3611; A61B 5/0826; A61B 5/4836; A61N 1/0551; A61N 1/36003; A61N 1/36139; A61N 1/36157; A61N 1/36171; A61N 1/36175; A61N 1/36178; A61N 1/36185; A61N 1/37217; A61N 1/3754; A61N 1/3787

(57) ABSTRACT

The present disclosure provides a neurostimulator implant for treating sleep apnea. The neurostimulator implant comprises an electrode lead and a stimulation controller. The electrode lead comprises a plurality of electrodes spaced along the electrode lead. The electrode lead is implantable in a subject such that at least some of the electrodes are implanted to stimulate a distal branch of the hypoglossal nerve. The stimulation controller is configured to independently control the plurality of electrodes.

### CROSS-REFERENCE TO RELATED APPLICATIONS

This application claims priority under 35 U.S.C. § 119(e) to U.S. Provisional Application No. 63/805,708 filed May 14, 2025, entitled NEUROSTIMULATOR IMPLANT AND METHOD FOR TREATING SLEEP APNEA, and under 35 U.S.C. § 119(b) to Republic of Ireland Application No. 2025/0231 filed May 29, 2025, the disclosures of which are incorporated herein by reference in their entirety for all purposes.

### BACKGROUND

There exist a number of treatments for sleep apnea, such as continuous positive airway pressure devices (CPAP), surgically implanted neurostimulators, oral mouthpieces, surgical procedures, and medicines. Generally, known devices have moderate efficacy with respect to reducing apnea-hypopnea index (AHI) and/or they are highly invasive devices procedures that can only be performed by surgical physicians under general anesthesia. While positive airway pressure (PAP) devices usually have high efficacy, many patients do not tolerate long-term use and potential users are unwilling to adopt it due to aversion or phobia to the system components. Additionally, neurostimulators and PAP devices on the market act primarily as standalone devices, capable of partially treating only Obstructive Sleep Apnea (OSA) or Central Sleep Apnea (CSA).

It is known for implanted neurostimulators to target the hypoglossal nerve to cause opening of the airway and tongue protrusion to mitigate sleep apnea. However, parts of the hypoglossal nerve also innervate muscles that cause tongue retrusion and/or closing of the airway, and it is challenging to precisely target only the parts of the hypoglossal nerve that cause tongue protrusion and opening of the airway.

### SUMMARY

According to a first aspect of the present invention there is provided a neurostimulator implant for treating sleep apnea, the neurostimulator implant comprising an electrode lead and a stimulation controller, wherein the electrode lead comprises a plurality of electrodes spaced along the electrode lead, the electrode lead being implantable in a subject such that at least some of the electrodes are implanted to stimulate a distal branch of the hypoglossal nerve, and wherein the stimulation controller is configured to independently control the plurality of electrodes.

In examples, the stimulation controller may be configured to control the plurality of electrodes to deliver charge-balanced biphasic pulses. In examples, the stimulation controller may be configured to control the plurality of electrodes to deliver pulse train stimulation. For example, the stimulation controller may be configured to control the plurality of electrodes to deliver different pulse train stimulation at different electrodes.

In examples, the stimulation controller may be configured to selectively operate the plurality of electrodes in at least one electrode array comprising a set of the plurality of electrodes. For example, the or each electrode array may comprise a bipolar electrode configuration or a tripolar electrode configuration. In examples, the stimulation controller may be configured to selectively operate the plurality of electrodes in a first electrode array and a second electrode array. For example, the stimulation controller may be configured to operate the first electrode array and the second electrode array with different stimulation parameters, including stimulation amplitude and frequency.

In examples, the plurality of electrodes may comprise a first set of electrodes and a second set of electrodes. The first set of electrodes may be arranged on the electrode lead to stimulate the distal branch of the hypoglossal nerve when implanted. The second set of electrodes may be arranged on the electrode lead to stimulate a geniohyoid muscle of the subject when implanted. In examples, the stimulation controller may be configured to control the first set of electrodes and the second set of electrodes to deliver different stimulation. In examples, the stimulation controller may be configured to control the electrodes to selectively stimulate the distal branch of the hypoglossal nerve with the first set of electrodes and the geniohyoid muscle with the second set of electrodes. In examples, the first set of electrodes may be arranged on the electrode lead to stimulate plural branches of the hypoglossal nerve when implanted.

In examples, the stimulation controller may be configured to control the first set of electrodes to define a first electrode configuration, and to control the second set of electrodes to a define a second electrode configuration, each electrode configuration comprising a bipolar electrode configuration or a tripolar electrode configuration.

In examples, the stimulation controller may be configured to control the electrodes to sequentially stimulate the hypoglossal nerve and the geniohyoid muscle during use. In other examples, the stimulation controller may be configured to control the electrodes to simultaneously stimulate the hypoglossal nerve and the geniohyoid muscle during use.

In examples, the neurostimulator implant may comprise a wireless power receiver for receiving wireless power from an external device, for example a wearable device. The wireless power receiver may be an RF wireless power receiver or a magnetic wireless power receiver.

In examples, the neurostimulator implant may comprise a battery.

In examples, the neurostimulator implant may further comprise a sensor for detecting a physiological parameter or movement of the subject. The stimulation controller may be configured to receive a sensor signal from the sensor and to control the plurality of electrodes based on the detected physiological parameter or movement. The physiological parameter may be a breathing phase and/or breathing rate of the subject. Additionally, or alternatively, the physiological parameter may be a heart rate or the blood oxyhemoglobin saturation percentage (SpO₂) of the subject.

In examples, the stimulation controller may be configured to generate stimulation signals based on a predetermined stimulation pattern.

According to a second aspect of the present invention, there is provided a method of treating sleep apnea in a subject, the method comprising: implanting a neurostimulator implant in the subject, the neurostimulator implant comprising an electrode lead and a stimulation controller, the electrode lead having a plurality of electrodes spaced along the electrode lead, wherein at least some of the electrodes are implanted to stimulate a distal branch of the hypoglossal nerve, and by the stimulation controller, independently operating the plurality of electrodes to stimulate the distal branch of the hypoglossal nerve of the subject.

In examples, the method may comprise, by the stimulation controller, controlling the plurality of electrodes to charge-balanced biphasic pulses.

In examples, the method may comprise, by the stimulation controller, controlling the plurality of electrodes to deliver pulse train stimulation.

In examples, the method may comprise, by the stimulation controller, controlling the plurality of electrodes to deliver different pulse train stimulation at different electrodes.

In examples, the method may comprise, by the stimulation controller, selectively operating the plurality of electrodes in at least one electrode configuration comprising a set of the plurality of electrodes. The or each electrode configuration may comprise a bipolar electrode configuration or a tripolar electrode configuration.

In examples, the method may comprise, by the stimulation controller, selectively operating the plurality of electrodes in a first electrode array and a second electrode array. The method may comprise, by the stimulation controller, operating the first electrode array and the second electrode array with different stimulation parameters, including stimulation amplitude and frequency.

In examples, the method may comprise, by the stimulation controller, operating the plurality of electrodes to define a first set of electrodes and a second set of electrodes, the first set of electrodes being implanted to stimulate the distal branch of the hypoglossal nerve when implanted, and the second set of electrodes being implanted to stimulate a geniohyoid muscle of the subject when implanted. In examples, the method may comprise, by the stimulation controller, controlling the first set of electrodes and the second set of electrodes to deliver different stimulation to the distal branch of the hypoglossal nerve and to the geniohyoid muscle. In examples, the method may comprise, by the stimulation controller, selectively stimulating the distal branch of the hypoglossal nerve with the first set of electrodes and the geniohyoid muscle with the second set of electrodes. In examples, the method may comprise, by the stimulation controller, controlling the first set of electrodes to define a first electrode configuration, and controlling the second set of electrodes to a define a second electrode configuration, each electrode configuration comprising a bipolar electrode configuration or a tripolar electrode configuration. The first set of electrodes may be implanted to stimulate plural branches of the hypoglossal nerve when implanted. In examples, the method may comprise, by the stimulation controller, controlling the first set of electrodes to selectively stimulate plural branches of the hypoglossal nerve.

In examples, the method may comprise sequentially stimulating the hypoglossal nerve and the geniohyoid muscle. Alternatively, the method may comprise simultaneously stimulating the hypoglossal nerve and the geniohyoid muscle.

In examples, the method may comprise receiving wireless power from an external device. Wireless power transfer may be RF or magnetic wireless power transfer.

In examples, the method may comprise detecting, by a sensor, a physiological parameter or movement of the subject. The method may further comprise, by the stimulation controller, controlling the electrodes based on the detected physiological parameter or movement.

According to a third aspect of the present invention there is provided a neurostimulator implant comprising: a housing portion having a wireless power receiver, an electrode lead comprising a plurality of electrodes, and a connecting lead extending between the housing portion and the electrode lead, wherein the connecting lead comprises a length such that the neurostimulator device is implantable in a subject with the housing portion disposed posterior to the ramus of the mandible of the subject with the connecting lead extending to a submandibular location, and the electrode lead being implanted proximate to a distal branch of the hypoglossal nerve.

In examples, the electrode lead may be implanted proximate to plural branches of the hypoglossal nerve.

In examples, the neurostimulator device is disposed in a pocket defined posterior to the ramus of the mandible of the subject. In examples, the neurostimulator device is disposed between muscle layers in the location posterior to the ramus of the mandible of the subject.

In examples, the connecting lead may have a length of between 5 cm and 15 cm, for example between 6 cm and 14 cm, for example between 7 cm and 13 cm, for example between 5 cm and 20 cm, for example between 6 cm and 18 cm, for example between 7 cm and 15 cm, for example between 8 cm and 12 cm, for example between 8 cm and 11 cm, for example between 8 cm and 10 cm.

In examples, the connecting lead may be integrally formed with the electrode lead. In other examples, the neurostimulator implant may comprise a connector for connecting the connecting lead to the electrode lead. In examples, the neurostimulator implant may additionally or alternatively comprise a connector for connecting the connecting lead to the housing portion.

The wireless power receiver may be an RF wireless power receiver or a magnetic wireless power receiver.

Features of the first aspect of the invention, described above, may be provided to the neurostimulator implant of the third aspect of the invention.

According to fourth aspect of the present invention, there is provided a method of implanting a neurostimulator implant, the neurostimulator implant comprising a housing portion having a wireless power receiver, an electrode lead comprising a plurality of electrodes, and a connecting lead extending between the housing portion and the electrode lead, the method comprising implanting: the housing portion in a position posterior to the ramus of the mandible of the subject, the connecting lead extending from the housing portion to a submandibular location of the subject, and the electrode lead being implanted proximate to a distal branch of the hypoglossal nerve.

In examples, the electrode lead may be implanted proximate to plural branches of the hypoglossal nerve.

In examples, the housing portion is implanted in a pocket defined posterior to the ramus of the mandible of the subject. In examples, the housing portion is implanted between muscle layers in the position posterior to the ramus of the mandible of the subject.

In examples, the connecting lead may be implanted along a jawline of the subject. In other examples, the connecting lead may extend across the submandibular area.

In examples, the connecting lead extends to a position proximate to the posterior edge of the mandibular symphysis. The electrode lead may extend in an anterior-posterior direction to position the plurality of electrodes proximate to the distal branch of the hypoglossal nerve.

In examples, the electrode lead may extend in an anterior-posterior direction to position the plurality of electrodes proximate to plural branches of the hypoglossal nerve.

In examples, the method may comprise: implanting the connecting lead such that it protrudes from the skin of the subject adjacent to the posterior edge of the mandibular symphysis, and subsequently implanting the electrode lead such that the plurality of electrodes are implanted proximate to the distal branch of the hypoglossal nerve.

In examples, implanting the electrode lead may comprise using an implant delivery device comprising a delivery sheath holding the electrode lead, the delivery sheath being moved percutaneously to carry the electrode lead to the position proximate to the distal branch of the hypoglossal nerve and then withdrawn.

In examples, the electrode lead may be implanted proximate to plural branches of the hypoglossal nerve.

In examples, the method may comprise implanting the connecting lead by tunnelling.

Wireless power transmission may be RF or magnetic wireless power transmission.

According to a fifth aspect of the present invention there is also provided a method of implanting a neurostimulator implant in a neck of a patient for treating sleep apnea, the neurostimulator implant comprising a housing portion and a flexible electrode lead connected thereto having at least one electrode, the method comprising:
advancing the electrode lead from a position proximal to a midline of the patient along a laterally extending insertion path to position the at least one electrode at a target anatomical location for neural stimulation.

The electrode lead and/or neurostimulator implant may be inserted at an insertion point. The insertion point may be proximal to or on the midline of the neck. The electrode lead may be progressed from the insertion point, along the insertion path, to position the at least one electrode of the electrode lead at the target anatomical location.

The target anatomical location may be on one lateral side of the neck. The target anatomical location may proximal to be the geniohyoid muscle and/or the genioglossus muscle and/or the hypoglossal muscle and/or the hypoglossal nerve. In particular, the target anatomical location may be proximal to the distal branch of the hypoglossal nerve and/or the medial branch of the hypoglossal nerve.

The target anatomical location may be between the geniohyoid muscle, the genioglossus muscle, the mylohyoid muscle and sublingual gland.

The electrode lead may comprise a plurality of electrodes. The electrode(s) may be located on an end (e.g., length segment/portion) of the electrode lead. After insertion, the end of the electrode lead may be close to or parallel with, and optionally overlapping, the distal branch of the hypoglossal nerve.

The electrode lead may comprise a first set of electrodes for stimulating a first muscle and a second set of electrodes for stimulating a second muscle. The electrode lead may comprise a first set of electrodes for stimulating a first muscle fibre group within a muscle and a second set of electrodes for stimulating a second fibre group within a muscle. For example, the electrodes may be positioned stimulate both the horizontal and the oblique fibres of the genioglossus muscle. Beneficially, this may achieve both downwards movement and protrusion of the tongue.

The neurostimulator implant may comprise a connecting lead extending between the housing portion and the electrode lead.

According to a sixth aspect of the present invention there is provided a neurostimulator implant for treating sleep apnea in a subject, the neurostimulator implant comprising: a stimulation controller, and an electrode lead having a plurality of electrodes, wherein the stimulation controller is connected with the plurality of electrodes and configured to generate a stimulation signal for stimulating a distal branch of a hypoglossal nerve of the subject during use, wherein the stimulation signal comprises a pulse train comprising a plurality of charge-balanced biphasic pulses, wherein each charge-balanced biphasic pulse comprises an amplitude of between 0.1 and 20 mA, and a pulse width of between 1 and 1000 microseconds, wherein each pulse train comprises an intratrain frequency of between 50 and 1000 Hz, and wherein an intertrain frequency is between 1 and 50 Hz.

In examples, the stimulation controller may be connected with the plurality of electrodes and configured to generate a stimulation signal for stimulating plural branches of the hypoglossal nerve of the subject during use.

In examples, each charge-balanced biphasic pulse comprises an active charge-balanced biphasic pulse comprising an anodic phase and a cathodic phase, the anodic and cathodic phases having equal amplitude and pulse width.

In examples, each charge-balanced biphasic pulse comprises a passive charge-balanced biphasic pulse comprising an anodic phase and a cathodic phase, the anodic and cathodic phases having non-equal amplitude and pulse width.

In examples, each charge-balanced biphasic pulse comprises an amplitude of between 0.5 and 15 mA, for example between 0.5 and 10 mA, for example between 1 and 8 mA, for example between 1 and 5 mA, for example between 1.5 and 5 mA, for example between 1.5 and 3.5 mA, for example between 1.8 and 3.2 mA, for example between 2 and 3 mA, for example about 2.5 mA.

In examples, each charge-balanced biphasic pulse comprises a pulse width of between 50 and 800 microseconds, for example between 100 and 600 microseconds, for example between 100 and 500 microseconds, for example between 100 and 600 microseconds, for example between 150 and 600 microseconds, for example between 200 and 300 microseconds, for example about 250 microseconds.

In examples, the intratrain frequency is between 50 Hz and 500 Hz, for example between 50 Hz and 400 Hz, for example between 100 Hz and 400 Hz, for example between 100 Hz and 350 Hz, for example between 100 Hz and 300 Hz, for example between 120 Hz and 300 Hz, for example about 120 Hz, for example about 180 Hz, for example about 300 Hz.

In examples, the intertrain frequency is between 10 Hz and 50 Hz, for example between 10 Hz and 40 Hz, for example between 15 Hz and 40 Hz, for example between 20 Hz and 30 Hz, for example 10 Hz or 15 Hz, or 20 Hz, or 25 Hz or 30 Hz or 35 Hz, or 40 Hz, or 45 Hz or 50 Hz.

According to a seventh aspect of the present invention there is provided a method of treating sleep apnea in a subject, comprising:
implanting a neurostimulator implant comprising a plurality of electrodes and a stimulation controller, the electrodes being implanted to stimulate a distal branch of a hypoglossal nerve of the subject,
by the stimulation controller, generating a stimulation signal for the electrodes and stimulating the distal branch of the hypoglossal nerve based on the stimulation signal,
wherein the stimulation signal comprises a plurality of pulse trains, each pulse train comprising a plurality of charge-balanced biphasic pulses, wherein each charge-balanced biphasic pulse comprises an amplitude of between 0.1 and 20 mA, and a pulse width of between 1 and 1000 microseconds, wherein each pulse train comprises an intratrain frequency of between 50 and 1000 Hz, and wherein an intertrain frequency is between 1 and 50 Hz.

In examples, each charge-balanced biphasic pulse comprises an active charge-balanced biphasic pulse comprising an anodic phase and a cathodic phase, the anodic and cathodic phases having equal amplitude and pulse width.

In examples, each charge-balanced biphasic pulse comprises a passive charge-balanced biphasic pulse comprising an anodic phase and a cathodic phase, the anodic and cathodic phases having non-equal amplitude and pulse width.

In examples, each charge-balanced biphasic pulse comprises an amplitude of between 0.5 and 15 mA, for example between 0.5 and 10 mA, for example between 1 and 8 mA, for example between 1 and 5 mA, for example between 1.5 and 5 mA, for example between 1.5 and 3.5 mA, for example between 1.8 and 3.2 mA, for example between 2 and 3 mA, for example about 2.5 mA.

In examples, each charge-balanced biphasic pulse comprises a pulse width of between 50 and 800 microseconds, for example between 100 and 600 microseconds, for example between 100 and 500 microseconds, for example between 100 and 600 microseconds, for example between 150 and 600 microseconds, for example between 200 and 300 microseconds, for example about 250 microseconds.

In examples, the intratrain frequency is between 50 Hz and 500 Hz, for example between 50 Hz and 400 Hz, for example between 100 Hz and 400 Hz, for example between 100 Hz and 350 Hz, for example between 100 Hz and 300 Hz, for example between 120 Hz and 300 Hz, for example about 120 Hz, for example about 180 Hz, for example about 300 Hz.

In examples, the intertrain frequency is between 10 Hz and 50 Hz, for example between 10 Hz and 40 Hz, for example between 15 Hz and 40 Hz, for example between 20 Hz and 30 Hz, for example 10 Hz or 15 Hz, or 20 Hz, or 25 Hz or 30 Hz or 35 Hz, or 40 Hz, or 45 Hz or 50 Hz.

In examples, the electrodes may be implanted to stimulate plural distal branches of the hypoglossal nerve of the subject.

According to an eighth aspect of the present invention, there is provided a method of implanting a neurostimulator implant in a subject for treating sleep apnea, the neurostimulator implant comprising a housing portion and a flexible electrode lead extending from the housing, the method comprising:
providing a carrier element holding the electrode lead,
advancing the carrier element and the electrode lead subcutaneously from a medial submandibular location in a superior direction until a tip of the carrier element is in contact with, or proximal to, the mandibular symphysis,
rotating and advancing the carrier element and the electrode lead along a posterior-lateral-superior insertion path until an electrode of the electrode lead is disposed proximal to a distal branch of the hypoglossal nerve of the subject, and
removing the carrier element from the subject to implant the electrode lead.

In examples, the method further comprises implanting the housing portion in the submandibular area of the subject. In other examples, the housing portion is elongate and the electrode lead extends from a first end of the housing portion, and wherein the housing portion is implanted such that the first end of the housing portion is disposed medially of the other end of the housing portion.

In examples, the housing portion is implanted in a posterior-lateral orientation.

It will be appreciated that features of the different aspects of the present invention described above may be combined.

In examples, the neurostimulator implant described above may be provided in addition to one or more further medical implants, in particular one or more further neurostimulator implants. The methods described above may further comprise sensing or stimulation by the one or more further medical implants.

The further medical implant(s) may include sensor implants for detecting a physiological parameter or movement of the subject, and/or for detecting an apneic event. In examples, the further medical implant(s) may include one or more further neurostimulator implants, for example one or more further neurostimulator implants implanted to target the phrenic nerve and/or an ansa cervicalis nerve of the subject. The neurostimulator implant and further medical implants may be in communication with each other (either directly, or via the external device) to enable combined sensing and/or stimulation therapy. Stimulation of the hypoglossal nerve, genioglossus muscle, phrenic nerve and/or an ansa cervicalis may be in response to a detected physiological parameter, patient movement, or classification of sleep apnea as described above.

In other examples, the system may include a continuous positive air pressure, CPAP, device, and the methods may include operating the CPAP device along with the neurostimulator implant. Operation of the CPAP device may be in response to a detected physiological parameter or movement of the subject. The operation of the CPAP device may be in response to a detected apneic event. The operation of the CPAP device may be synchronized with the stimulation of the hypoglossal nerve and/or the genioglossus muscle.

In examples, the method may further comprise controlling the or each neurostimulator implant, and optionally the CPAP device, based on the detected at least one physiological parameter and/or movement of the subject.

### BRIEF DESCRIPTION OF THE DRAWINGS

Embodiments of the invention will be described, by way of example only, with reference to the accompanying drawings, in which:

FIG. 1 illustrates an implantable neurostimulator implant.

FIG. 2 illustrates a schematic diagram of the neurostimulator implant.

FIG. 3A illustrates head and neck anatomy of a subject in the sagittal plane.

FIG. 3B illustrates an anatomical implantation site of the neurostimulator implant in the sagittal plane.

FIG. 3C illustrates the anatomical implantation site of the neurostimulator implant from the posterior side of the submandibular area.

FIG. 4A and FIG. 4B illustrate example implantation profiles for the neurostimulator implant.

FIG. 5A illustrates an example insertion point and insertion orientation for implanting the neurostimulator implant.

FIG. 5B illustrates a bottom view of the insertion point and insertion orientation shown in FIG. 5A.

FIG. 5C illustrates a side view of the insertion point and insertion orientation shown in FIG. 5A.

FIG. 6A and FIG. 6B illustrate alternative implantation sites of the neurostimulator implant.

FIG. 7 illustrates an alternative neurostimulator implant.

FIG. 8 illustrates an example pulse stimulation signal.

FIG. 9 illustrates an example charge balanced pulse stimulation signal.

FIG. 10 illustrates a further example charge balanced pulse stimulation signal.

FIG. 11 illustrates a further example charge balanced pulse stimulation signal.

FIG. 12 illustrates an example pulse train stimulation.

FIG. 13 illustrates a further example pulse train stimulation.

FIG. 14 illustrates a further example pulse train stimulation.

FIG. 15 illustrates a bar graph of the mean Apnea-Hypopnea Index (AHI) before treatment (pre-treatment) and at 4-days post-implant obtained from polysomnography of 10 subjects diagnosed with moderate-to-severe apnea.

### DETAILED DESCRIPTION

To easily identify the discussion of any particular element or act, the most significant digit or digits in a reference number refer to the figure number in which that element is first introduced.

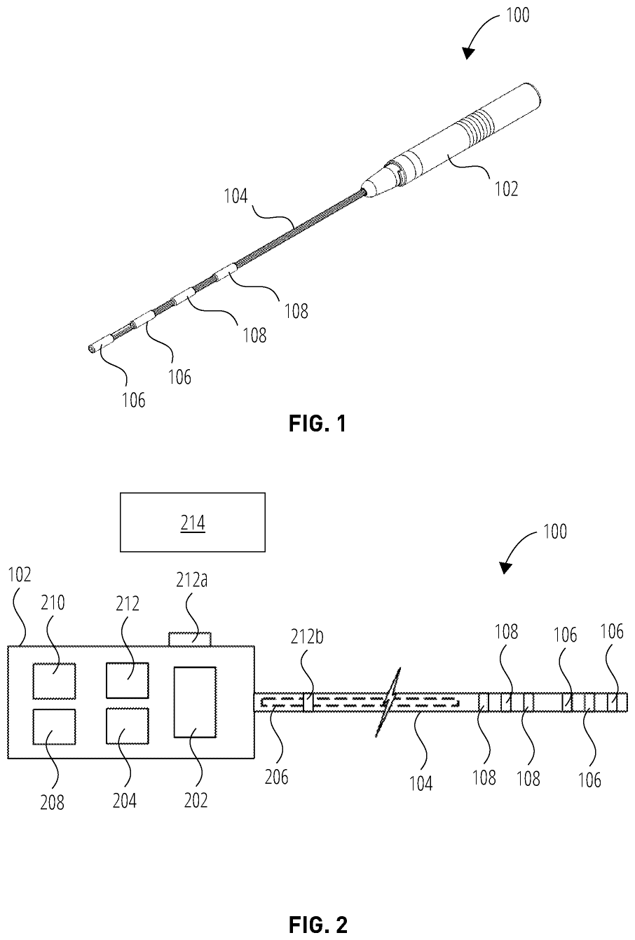

FIG. 1A illustrates a neurostimulator implant 100. The neurostimulator implant 100 comprises a housing portion 102 and an electrode lead 104. As described further hereinafter, the housing portion 102 houses an electronics assembly of the neurostimulator implant 100. For example, the electronics assembly may include a printed circuit board (PCB), a wireless communications receiver/transmitter, a wireless power receiver, and/or sensor electronics. In examples, the housing portion 102 is hermetically sealed. The housing portion 102 is preferably formed of a ceramic tube with end caps and a feedthrough to connect lead wires of the electrode lead 104 to the electronics assembly in the housing portion 102.

The electrode lead 104 extends from the housing portion 102 and is flexible. The electrode lead 104 includes at least one electrode 106, in this example four electrodes 106, 108. In other examples, the electrode lead 104 may comprise one, two, three, five, six, or more electrodes. The electrodes 106, 108 are spaced along the length of the electrode lead 104, particularly at an end of the electrode lead 104 opposite to the housing portion 102. The electrodes 106, 108 are arranged in an electrode array. The electrodes 106, 108 are connected to the electronics within the housing portion 102. The electrodes 106, 108 are metal, preferably a platinum-iridium alloy or titanium.

In examples, the electrodes 106, 108 are operated by the electronics assembly housed in the housing portion 102, to provide electrical stimulation to stimulate a nerve and/or muscle of the subject as described further hereinafter. In particular, a stimulation controller may be provided in the housing portion 102 as described hereinafter.

In examples, the housing portion 102 may have an outer diameter of less than about 5 millimetres, for example between about 1 millimetre and about 5 millimetres, for example between about 1 millimetre and about 3 millimetres, for example about 3 millimetres. The housing portion 102 may have a length of up to about 30 millimetres, for example up to about 25 millimetres, for example up to about 20 millimetres, for example about 20 millimetres. In examples, the electrode lead 104 may have a diameter of between about 0.3 millimetres to about 1.5 millimetres, for example between about 0.5 millimetres and 1.3 millimetres, for example about 0.8 millimetres. The electrode lead 104 may have a length of up to about 200 millimetres, for example up to about 150 millimetres, for example up to about 120 millimetres, for example about 120 millimetres.

The electrodes 106, 108 may each have a length (along the electrode lead 104) of up to about 15 millimetres, for example up to about 10 millimetres, for example up to about 5 millimetres, for example about 3 millimetres. The electrodes 106, 108 may be spaced from each other along the electrode lead 104 by at least 2 millimetres, for example at least 3 millimetres, for example at least 5 millimetres.

It will be appreciated that the dimensions of the housing portion 102 would correspond to the size of the electronics assembly within the housing portion 102, and the length of the electrode lead 104 would correspond to the anatomy surrounding the targeted nerve, so a shorter or longer electrode lead 104 may be appropriate depending on the depth of the nerve within the subject's tissue.

The electrodes 106, 108 are arranged in two sets: a first set of electrodes 106 and a second set of electrodes 108. As described below, when implanted, the first set of electrodes 106 may provide neural stimulation, particularly to the hypoglossal nerve, and the second set of electrodes 108 may provide muscle stimulation, particularly to the geniohyoid muscle. Each set of electrodes 106, 108 comprises at least two electrodes such that each set can form at least one bipolar or tripolar electrode configuration.

FIG. 2 shows a schematic diagram of the neurostimulator implant 100 of FIG. 1. As shown, the neurostimulator implant 100 includes the housing portion 102 and the electrode lead 104 with electrodes 106, 108 as described above. The electrodes 106, 108 are arranged as a first set of electrodes 106 and a second set of electrodes 108. The first set of electrodes 106 is located more distally on the electrode lead 104 than the second set of electrodes 108. That is, the second set of electrodes 108 are disposed between the first set of electrodes 106 and the housing portion 102, or the first set of electrodes 106 are closer to the distal end of the electrode lead 104 than the second set of electrodes 108. In this way, when implanted the first set of electrodes 106 can be implanted at a greater depth than the second set of electrodes 108. As described further below, in this way the first set of electrodes 106 can target the distal branch of the hypoglossal nerve and the second set of electrodes 108 can be implanted for stimulation of the geniohyoid muscle.

As illustrated in FIG. 2, the housing portion 102 houses a stimulation controller 202. The stimulation controller 202 is connected with the electrodes 106, 108 and configured to generate electrical pulses for neural and/or muscular stimulation by the electrodes 106, 108. The stimulation controller 202 is configured to individually control each of the electrodes 106, 108. This allows the stimulation controller 202 to control the electrodes 106, 108 to form different groups, for example one or more monopolar, bipolar, or tripolar electrode configurations. As described in detail below, the stimulation controller 202 may control the electrodes 106, 108 to deliver pulse-based stimulation to the nerve and/or muscle of the subject. The pulse-based stimulation may include at least one of a series of individual pulses, pulse train stimulation, and the like. The stimulation controller 202 may be configured to control the electrodes 106, 108 in different monopolar, bipolar or tripolar configurations or sets and deliver different pulses or pulse trains to each configuration or set.

The stimulation controller 202 may comprise a processor which may in turn comprise processing means and memory means. The processing means may be one or more electronic processing devices which operably execute computer-readable instructions. The memory means may be one or more memory devices. The memory means is electrically coupled to the processing means. The memory means is configured to store instructions, and the processing means is configured to access the memory means and execute the instructions stored thereon. The processor may comprise an input means and an output means. The input means may comprise an electrical input of the processor. The output means may comprise an electrical output of the processor. The input may be arranged to receive an electrical signal, for example from the other components of the neurostimulator implant 100. The output may be arranged to output an electrical signal, for example for controlling output of an electrical pulse at the electrodes 106, 108.

The housing portion 102 also houses a wireless power receiver 204. The wireless power receiver 204 is configured to receive wireless power from an external device 214. The wireless power transfer mode may be RF or magnetic. An antenna 206 may be provided in at least a part of the electrode lead 104 for receiving wireless power. The antenna may additionally or alternatively be formed in the housing portion 102. More than one power receiving antenna may be provided in the neurostimulator implant 100. The wireless power receiver 204 is preferably an induction coupling wireless power system, but may alternatively be a capacitive coupling system. The wireless power receiver 204 and antenna 206 are preferably configured for near-field wireless power transfer.

In the illustrated example the neurostimulator implant 100 also optionally includes a communications transceiver 210 for wireless communicating with the external device 214. The communications transceiver 210 and external device 214 may exchange control data, for example instructions for operating the stimulation controller 202 and/or power transmission information. The communications transceiver 210 may be connected to the antenna 206 for receiving/transmitting control data. Alternatively, a separate antenna may be provided for communications.

The external device 214 may be provided in a wearable garment, which may be strapped or adhered to the subject so as to overlie the implantation site. In particular, the external device 214 may be secured at the underside of the chin of the subject, or integrated into a scarf or collar or other similar garment worn on the neck of the subject. Alternatively, the external device 214 may be integrated into another device that is located close to the subject's head and/or neck during sleep, for example in bedding, specifically in a pillow or sheet. In this way, the external device 214 is close enough to the implanted neurostimulator implant 100 for wireless power transfer and/or wireless communications.

In the illustrated example the housing portion 102 also optionally houses a battery 208. The battery 208 may be charged by the wireless power receiver 204 and may store charge for powering the neurostimulator implant 100. The battery 208 may store enough charge to operate the neurostimulator implant 100 for at least one stimulation cycle. In examples, the battery 208 may have a capacity of at least 50 mAh, for example 50-100 mAh, for example about 75 mAh. In examples without a battery 208, the wireless power receiver 204 may directly power the stimulation controller 202 and other functions. A capacitor or other small power store may be provided for smoothing the power provided to the stimulation controller 202.

In the illustrated example the neurostimulator implant 100 also optionally includes a sensor 212. The sensor 212 is connected with at least one sensor electrode 212a, 212b. The sensor electrode 212a may be located on the housing portion 102 and/or the sensor electrode 212b may be located on the electrode lead 104. Preferably, more than one sensor electrode 212a, 212b is provided, either on the housing portion 102, or on the electrode lead 104, or on both. The sensor 212 may detect at least one of: a respiration rate of the subject; an electromyograph, EMG, of the subject, an electrocardiogram, ECG, of the subject, a blood oxygen saturation of the subject, a body temperature of the subject, a heart rate of the subject, and/or a blood pressure of the of the subject. In other examples, the sensor 212 may additionally or alternatively detect a movement of the subject, for example a respiratory movement of the subject, a mandibular movement of the subject, a thoracic movement of the subject, and a diaphragm movement of the subject. The sensor 212 may comprise a bioimpedance sensor, a biopotential sensor, a blood oxygen saturation sensor, an accelerometer and/or an ECG sensor.

Biosensors in the neurostimulator implant may detect signs of inspiration to trigger device activation and or general patient diagnostics. Such biosensors may include bioimpedance or biopotential based sensing devices whose respective signals are converted to ECG, EMG, EDA formats for interpreting heart rate, blood pressure, percentage of blood oxyhemoglobin saturation and/or electromyographic detection of inspiratory muscle contraction. The neurostimulator implant 100 may communicate with an external wearable device or an external power unit to combine data sets and/or trigger device activation in synchronisation with the subject's natural respiration rate.

In other examples the neurostimulator implant 100 may be part of a system with an external sensor. The external sensor may be configured to detect a respiration rate of the subject; an electromyograph, EMG, of the subject, an electrocardiogram, ECG, of the subject, a blood oxygen saturation of the subject, a body temperature of the subject, a heart rate of the subject, and/or a blood pressure of the of the subject. In other examples, the external sensor may additionally or alternatively detect a movement of the subject, for example a respiratory movement of the subject, a mandibular movement of the subject, a thoracic movement of the subject, and a diaphragm movement of the subject, and/or an apnea event. The external sensor may comprise a bioimpedance sensor, a biopotential sensor, a blood oxygen saturation sensor, an accelerometer and/or an ECG sensor.

In some examples the external sensor may comprise a mmWave radar sensor for detecting movement of the subject's chest in order to detect a respiration phase and/or rate and/or an apnea event. In some examples the external sensor may comprise an RF-based sensor configured to use channel variations (phase shifts, amplitude fluctuations) to detect movement of the subject's chest in order to detect a respiration phase and/or rate and/or an apnea event. Such an RF-based sensor may be arranged to transmit RF signals towards, or nearby, the subject in their sleeping location. The RF-based sensor may operate in Wi-Fi, UWB, or sub-GHz frequencies. In other examples the external sensor may be magnetic or inductive sensors. In particular, magnetic elements or inductive coils may be placed on the subject (e.g., on the thorax of the subject) and the external sensor may be configured to detect changes in magnetic field and/or coil inductance caused by movement of the magnetic elements or inductive coils to detect movement of the subject's chest in order to detect a respiration phase and/or rate.

The external sensor may be provided on a wearable to be worn by the subject, or may be provided separate to the subject (e.g., located nearby the sleeping location of the subject).

The stimulation controller 202 may be configured to receive a sensor signal from the external sensor and to control the plurality of electrodes based on the detected physiological parameter or movement or based on the detection of an apnea event. The physiological parameter may be a breathing phase and/or breathing rate of the subject. Additionally, or alternatively, the physiological parameter may be a heart rate or the blood oxyhemoglobin saturation percentage (SpO₂) of the subject. In some examples the stimulation controller 202 may be configured to dynamically control the stimulation to follow the detected respiration rate and phase of the subject. Additionally or alternatively, the stimulation controller 202 may be configured to control stimulation based on the detection of an apnea event, for example in response to detecting or predicted onset of an apnea event.

Information from the sensors described above (integrated and/or external sensors) may be used to provide health information to the subject, for example sleep cycle information, apnea event information and the like. In particular, sensor data may be transmitted to an external device, for example a subject's computing device (e.g., smartphone) for use in health tracking.

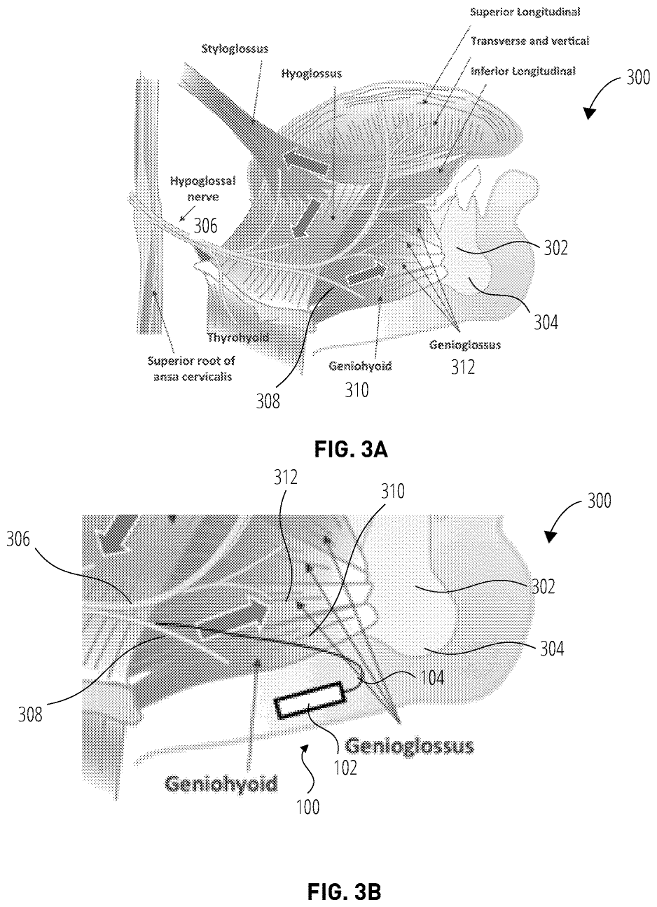

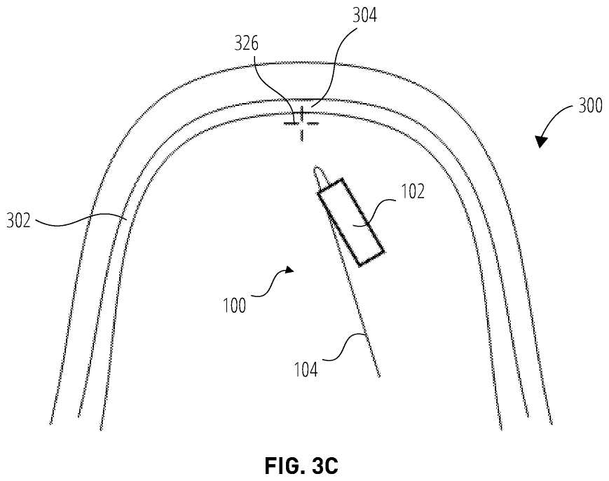

FIG. 3A illustrates the anatomy of a subject 300 in their head and neck region. FIG. 3A particularly shows the hypoglossal nerve 306 and associated muscles that are innervated by the hypoglossal nerve 306—in particular the genioglossus muscle 312. Also illustrated is the geniohyoid muscle 310, which is innervated by the first cervical nerve (C1), not illustrated, which travels alongside the hypoglossal nerve 306 along at least some of its length.

As illustrated, the hypoglossal nerve 306 includes a distal branch 308 that extends into, and innervates, the genioglossus muscle 312. The distal branch 308 of the hypoglossal nerve 306 may be termed the hypoglossal nerve branch.

The geniohyoid muscle 310 is important for breathing and treat sleep apnea. In particular, contraction of the geniohyoid muscle 310 protrudes the acts on the hyoid to open the subject's airway. Contraction of the genioglossus muscle 312 can cause tongue protrusion, which is also beneficial for treating sleep apnea.

It is therefore beneficial to be able to stimulate both the geniohyoid muscle 310 and the genioglossus muscle 312 for treating sleep apnea.

FIG. 3A also shows the mandible 302 of the subject 300, and the mandibular symphysis 304. The geniohyoid muscle 310 extends from the mandible 302 to the hyoid, and the genioglossus muscle 312 extends from the mandible 302 to the tongue.

FIG. 3B and FIG. 3C show the implantation site of the neurostimulator implant 100 in the subject 300. As shown, the neurostimulator implant 100 is implanted such that the housing portion 102 is located in subcutaneous tissue in the submandibular region of the subject 300. Subcutaneous tissues in the submandibular region may include any tissues in the space between the skin and the platysma muscle, or any intermuscular tissue between the platysma and the mylohyoid muscle or the digastric muscle. In some examples the housing portion 102 may be secured to a bone or ligament structure in the submandibular region, for example by suturing the housing portion 102 to the mandible or a tendon. The housing portion 102 may be anchored to the inner periosteum of the mandible. The housing portion 102 may include a hole or eyelet to facilitate this. Securing the housing portion 102 in this way can help prevent movement of the housing portion 102 post-implantation.

As shown in FIG. 3B, the electrode lead 104 extends towards, and into the geniohyoid muscle 310 and optionally also into the genioglossus muscle 312. An end of the electrode lead 104 is implanted proximate to the distal branch 308 of the hypoglossal nerve 306. Preferably, as shown, the end of the electrode lead 104 is close to parallel with, and overlapping, the distal branch 308 of the hypoglossal nerve 306.

In the implanted position, the first set of electrodes 106 (see FIG. 1 and FIG. 2) are implanted proximate to the distal branch 308 of the hypoglossal nerve 306, and the second set of electrodes 108 (see FIG. 1 and FIG. 2) are implanted in or on the geniohyoid muscle 310. In this way, the first set of electrodes 106 can be operated for neural stimulation of the distal branch 308 of the hypoglossal nerve 306, in order to cause innervation of the genioglossus muscle 312. Alternatively or additionally, the second set of electrodes 108 can be operated for direct muscle stimulation of the geniohyoid muscle 310. As described below, this arrangement of the electrodes 106, 108 allows operation of the neurostimulator implant 100 to be configured for the specific anatomy and implantation site of the subject.

The neurostimulator implant 100 may be implanted using a carrier element, for example a sheath, to carry the electrode lead 104 percutaneously to the implant position. Removal or traction of the sheath leaves the electrode lead 104 implanted. After implantation of the electrode lead 104, or simultaneously therewith, the housing portion 102 can be implanted through an incision in the skin in the submandibular area. As shown, the electrode lead 104 may be bent or kinked. This can provide for better positioning and orientation of the housing portion 102 (e.g., to sit within the subcutaneous tissue and/or to improve orientation of wireless power transfer) and may also help to prevent movement of the housing portion 102 pulling the electrode lead 104 out of position.

Applicant's patent application PCT/EP2021/085998 describes an implant delivery device that can be used for implanting the neurostimulator implant 100.

During implantation the exact positions of the electrodes on the electrode lead 104 with respect to the distal branch 308 of the hypoglossal nerve 306 and geniohyoid muscle 310 may vary, or may not be known precisely. In examples of the neurostimulator implant 100, the electrodes are individually controllable. This allows the best-positioned electrodes to be identified and used to deliver stimulation after implantation. In particular, by individually controlling each electrode it is possible to control the electrodes to select which are best positioned relative to the hypoglossal nerve 306 and the geniohyoid muscle 310. This allows the neurostimulator implant 100 to be configured based on the implant position to account for variations in the positions of the electrodes, and can also accommodate different subject's reaction to neural/muscle stimulation to improve the treatment of sleep apnea in the subject.

As shown in FIG. 3B and FIG. 3C, the electrode lead 104 is implanted in a posterior-lateral-superior orientation. In particular, the following method may be used to implant the neurostimulator implant 100.

A carrier element, for example a sheath, needle or catheter, may hold the electrode lead 104. The electrode lead 104 may be received within a lumen or slot of the carrier element. The carrier element and electrode lead 104 may be percutaneously inserted into the subject at an insertion point 326 located in the submandibular area proximate to a posterior edge of the mandibular symphysis 304, as shown in FIG. 3C.

From the insertion point 326 the carrier element and electrode lead 104 can be advanced in a superior direction towards the posterior edge of the mandibular symphysis 304. This advances the carrier element and electrode lead 104 through the subject's skin to an initial depth.

When a tip of the carrier element contacts the posterior edge of the mandibular symphysis 304, or is proximal thereto, the carrier element and electrode lead 104 can be rotated to align with an insertion path. In particular, the carrier element and electrode lead 104 can be rotated into a posterior-lateral-superior orientation. The carrier element and electrode lead 104 can then be advanced along a posterior-lateral-superior direction (insertion path) until the electrodes on the electrode lead 104 are positioned at the target anatomical location (described further below). The distance of this advancement may be between 3 cm and 6 cm, more often between 4 cm and 5 cm depending on the subject's anatomy.

The carrier element can then be removed, leaving the electrode lead 104 implanted with the electrodes at the target anatomical location.

In this way, the electrode lead 104 is implanted in an anterior-to-posterior, medial-to-lateral, and inferior-to-superior orientation in the submandibular area of the subject.

Once the electrode lead 104 has been implanted the housing portion 102 can be implanted in the subcutaneous tissue, for example through an incision.

As shown, the housing portion 102 is preferably oriented obliquely—non-parallel with the sagittal or frontal planes of the subject. In particular, the housing portion 102 is preferably implanted in a lateral-posterior orientation. The housing portion 102 is preferably approximately parallel with the skin of the subject, which may be beneficial for wireless power transfer if the housing portion 102 includes a power receiving antenna. The end of the housing portion 102 from which the electrode lead 104 extends is located approximately at or near the midline of the subject, and the housing portion 102 extends laterally and posteriorly, and approximately parallel with the skin of the subject. This advantageously allows the housing portion 102 to be implanted away from sensitive anatomical features, including the subject's trachea.

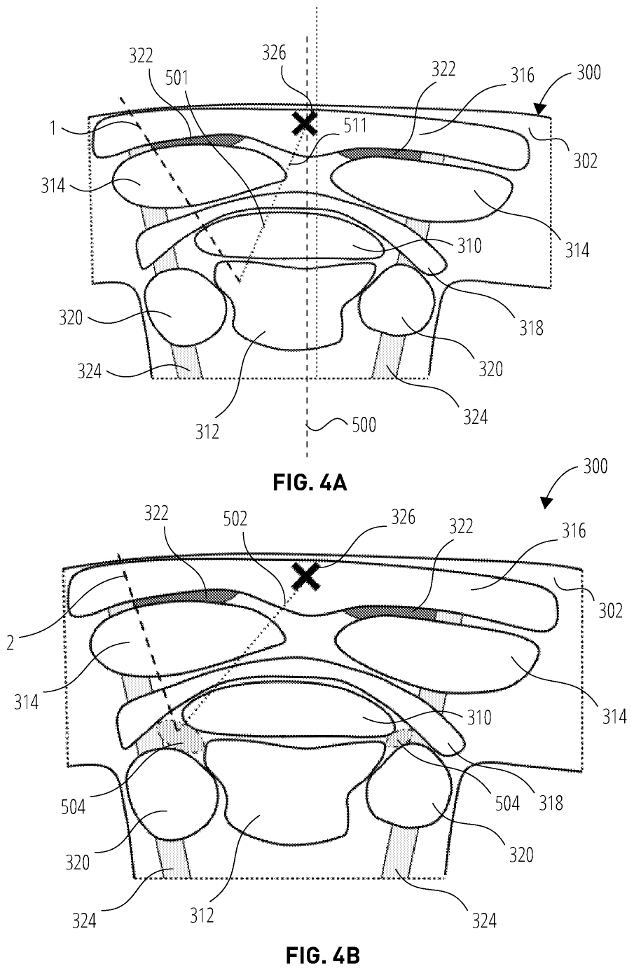

FIG. 4A and FIG. 4B illustrate example insertion paths for the electrode lead. As described above, during implantation the electrode lead is advanced along a posterior-lateral-superior insertion path to position the electrodes of the electrode lead at a target anatomical location. For treating sleep apnea, the target anatomical location is such that the electrodes are positioned proximal to the distal branch of the hypoglossal nerve and/or in the geniohyoid muscle. FIG. 4A shows a first insertion path 501 for reaching the target anatomical location, and FIG. 4B shows a second insertion path 502 for reaching the target anatomical location. It will be appreciated that after implantation the electrode lead remains approximately along the insertion path 501, 502.

FIG. 4A and FIG. 4B both show a scan-view of the submandibular and neck region of a subject. FIG. 4A and FIG. 4B show the mandible 302 shown at the top and the subcutaneous tissue 316 of the submandibular area, the digastric muscles 314, the geniohyoid muscle 310, the mylohyoid muscle 318, the genioglossus muscle 312, the sublingual glands 320, the submandibular salivary glands 322, and the two jugular veins 324 along each side of the neck. The subcutaneous tissue 316 in the submandibular area may include any tissues in the space between the skin and the platysma muscle, or any intermuscular tissue between the platysma and the mylohyoid muscle 318 or the digastric muscle 314. FIG. 4A and FIG. 4B also both show the insertion point 326, which is the same as illustrated in FIG. 3C—aligned with the posterior edge of the mandibular symphysis.

In the example of FIG. 4A the insertion path 501 extends from the insertion point 326 in a posterior-lateral-superior direction. The insertion path 501 extends to a lateral portion of the geniohyoid muscle 310 and to a lateral portion of the genioglossus muscle 312.

In this example the target anatomical location (where the electrodes are implanted) extends across the geniohyoid muscle 310 and the genioglossus muscle 312. In this way, electrodes on the electrode lead may be implanted proximate to the distal branch of the hypoglossal nerve. Preferably, a part of the electrode lead may be close to or parallel with, and overlapping, the distal branch of the hypoglossal nerve.

Once the electrode lead is implanted along the insertion path 501, some of the electrode(s) may be used to stimulate the hypoglossal nerve to innervate the genioglossus muscle 312. Additionally or alternatively, other electrodes may be used to directly stimulate the geniohyoid muscle 310. Dual stimulation of the hypoglossal nerve and the geniohyoid muscle 310 may be preferable to stimulation of a single nerve, muscle or muscle fibre group. For example, stimulating both the geniohyoid muscle 310 and the genioglossus muscle (via the hypoglossal nerve) may be more effective than single muscle stimulation in treating certain conditions such as sleep apnea.

In one example, a first set of electrodes may be implanted proximate to the distal branch of the hypoglossal nerve, and a second set of electrodes may be implanted in or on the geniohyoid muscle 310. In this way, the first set of electrodes can be operated for neural stimulation of the distal branch of the hypoglossal nerve, in order to cause innervation of the genioglossus muscle 312. Alternatively, or additionally, the second set of electrodes can be operated for direct muscle stimulation of the geniohyoid muscle 310.

In some examples, multiple electrodes may be positioned to stimulate the geniohyoid muscle 310. For example, the electrodes may be positioned on or in the geniohyoid muscle 310 and span across a distance of the geniohyoid muscle 310.

In some examples, electrodes may be implanted to stimulate the genioglossus muscle by direct muscle stimulation or by stimulating the hypoglossal nerve. For example, innervating both the horizontal and the oblique fibres of the genioglossus muscle may beneficially achieve both downwards movement and protrusion of the tongue. Such innervation may be achieved by ensuring at least one, or a pair, of the electrodes are positioned proximal to the medial branch of the hypoglossal nerve and at least one, or a pair, of the electrodes are positioned proximal to the distal branch of the hypoglossal nerve. In some examples, the electrodes may be positioned substantially parallel to the hypoglossal nerve to provide improved stimulation. In other examples, the two muscle groups of the genioglossus muscle may be stimulated directly (e.g., by positioning the electrodes on or in the genioglossus muscle and spanning across the two distinct muscle horizontal and oblique fibre groups).

After insertion, the electrode positioning may be tested e.g., by selecting different pairs of electrodes and applying electrical stimulation therebetween. The electrodes which achieved the most effective muscle response may then be selected for use during treatment.

FIG. 4B shows a further example insertion path 502 where the electrode lead is extended to a different target anatomical location. In this example, the target anatomical location 504 is between the geniohyoid muscle 310, the genioglossus muscle 312, the mylohyoid muscle 318 and sublingual gland 320. Stimulation of this target anatomical location 504 may be particularly effective for the treatment of sleep apnea.

In this example, similar to the example of FIG. 4A, the insertion path 502 extends from the insertion point 326 in a posterior-lateral-superior direction.

After insertion of the neurostimulator implant, at least one of the electrodes may be positioned in this target anatomical location 504. In some examples, after insertion all of the electrodes may be positioned in this target anatomical location 504. In other examples, after insertion, at least one of the electrodes may be positioned in the target anatomical location 504 indicated in FIG. 4B and at least one of the electrodes may be positioned on or in the geniohyoid muscle 310.

For each of the different target anatomical locations the insertion path extends from a medial position, laterally to the target anatomical location. This may be known as a medial-to-lateral approach. Such an approach may be preferable to a more conventional lateral-to-medial approach because it better avoids delicate anatomical features, including the submandibular salivary gland 322 and the jugular veins 324.

For comparison, a lateral-to-medial insertion path is illustrated in each of FIG. 4A and FIG. 4B. FIG. 4A shows lateral-to-medial insertion path 1, and FIG. 4B shows lateral-medial insertion path 2.

As shown such lateral-to-medial insertion paths 1, 2 may lie close to either or both the jugular vein 324 and the submandibular salivary gland 322. Such an approach may risk damaging these structures during insertion which can risk the subject's health and potentially lead to complications due to glandular damage. Due to the risk of compromising the jugular vein 324 or the carotid artery, such a procedure may have to be carried out in a surgical setting to ensure patient safety.

In addition, the insertion position for these approaches may be difficult to consistently locate on different subjects with different anatomy as there are no closely neighbouring anatomical landmarks that are consistently positioned for all subjects.

In contrast, insertion paths 501, 502 described above better avoid the sensitive anatomical locations such as the jugular vein 324 and the submandibular salivary gland 322, thereby improving patient outcomes and safety. In addition, the implantation method described above uses the posterior edge of the mandibular symphysis as a landmark to determine the insertion point 326 and the starting point of the insertion paths 501, 502, leading to improved accuracy and repeatability across different subjects.

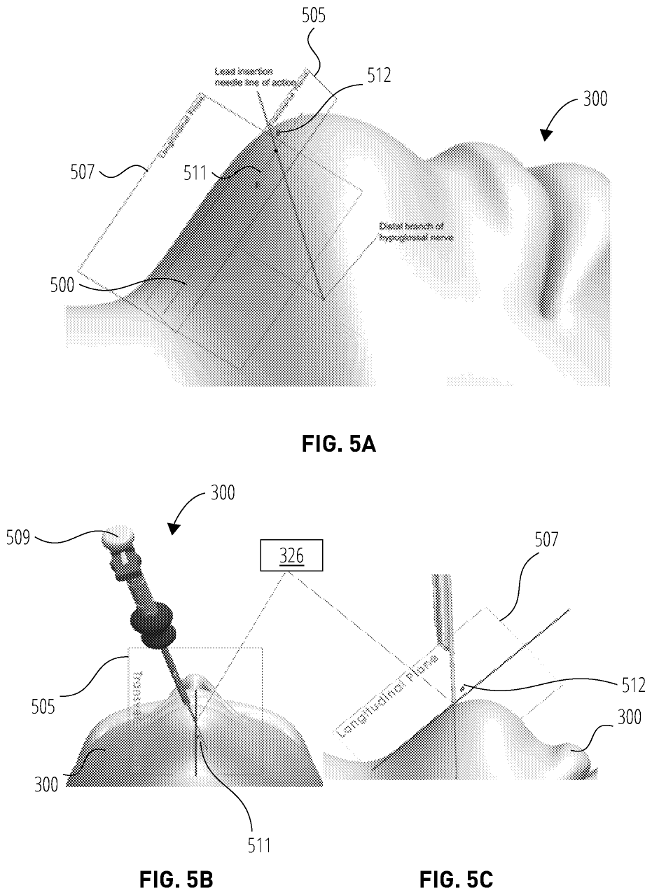

FIG. 5A to FIG. 5C illustrate an example delivery device 509 being used to implant the electrode lead along the insertion path 501 or insertion path 502.

As shown, the delivery device 509 includes a needle, which is holding the electrode lead of the neurostimulator implant. The needle penetrates the subject's skin at an insertion point 326 in the submandibular area, approximately aligned with the posterior edge of the mandibular symphysis of the subject 300. As described above, after initially advancing the needle in a superior direction, the delivery device 509 is rotated to the orientation, which is illustrated in FIG. 5A, FIG. 5B and FIG. 5C, with the needle directed in a posterior-lateral-superior orientation. The 509 and needle are then advanced along the insertion path 501, 502 until the target anatomical location is reached.

As shown in FIG. 5A, the orientation of the insertion path 501, 502 may be defined by a first angle 511 in a transverse plane 505. The transverse plane 505 may be approximately the muscular plane of the patient's chin when the neck is in a hyperextended position. The first angle 511 may be positive or negative, depending on whether the left or right hypoglossal nerve is being targeted. The first angle 511 is the angular offset from midline 500 (i.e., the median, or sagittal plane of the subject 300) in the transverse plane.

The insertion orientation is also defined by a second angle in a longitudinal plane. The longitudinal plane 507 is the patient's median (sagittal) plane. The second angle is the angular offset from the transverse plane in the longitudinal plane.

In examples, the first angle 511 may be between about ±5 degrees and about ±30 degrees, for example between about ±8 degrees and about ±25 degrees, for example between about ±10 degrees and about ±23 degrees, for example between about ±10 degrees and about ±20 degrees. In one particular example, the first angle 511 may be about ±22 degrees, in particular about ±21.8 degrees.

In examples, the second angle 512 may be between about 40 degrees and about 65 degrees, for example between about 45 degrees and about 55 degrees, for example between about 40 degrees and about 50 degrees. In particular example, the second angle 512 may be between about 47 degrees and about 49 degrees, in particular about 48.1 degrees.

It will be appreciated however that the first angle 511 and the second angle 512 may be different for different patients, and the first angle 511 and the second angle 512 may be determined for a particular patient in advance of the implantation procedure, for example based on an ultrasound scan or other scan information. As alternative to ultrasound scans, fluoroscopy, CT scanning or MRI scanning may be used. In some examples scan information may be used in a stereotactic surgical process, where scan information is used to construct a 3D point model of the subject and the target implant location. In such examples the implantation may be robotically performed.

In some examples, the insertion depth is also determined. The insertion depth may be approximately the distance from the skin to a branch of the hypoglossal nerve 306 (e.g., the distal branch 308). If the insertion depth is determined, it may be based on the below equations defined by Benbassat et al (Benbassat B, Cambronne C, Gallini A, Chaynes P, Lauwers F, de Bonnecaze G. The specific branches leading to the genioglossus muscle: three dimensional localisation using skin reference points.

L=\sqrt{(Y)²+(0.6 GH)²}

Y=\sqrt{(0.5 GH)²+(0.2 GH)²}
Where: L is the insertion depth. GH is a measured variable, defined as the distance from the posterior edge of the mandibular symphysis to the hyoid bone along the midline (in the transverse plane).

In other examples, the insertion depth may be determined in advance of the implantation procedure based on a medical image, for example an X-ray, ultrasound scan, CT scan, or MRI scan. In other examples, the operator may use a scanner, in particular an ultrasound scanner such as a doppler ultrasound, to monitor the insertion depth and position and to confirm the proximity of the implant to vascular structures during insertion. In other examples, the insertion depth is not determined, and instead the operator may insert the delivery device 509 to a predetermined depth. In other examples, the insertion depth may be tested and/or verified by generating stimulation signals and testing a reaction of the patient to the stimulation signal, and/or by using bioimpedance sensing to detect when the electrodes are implanted close to the nerve.

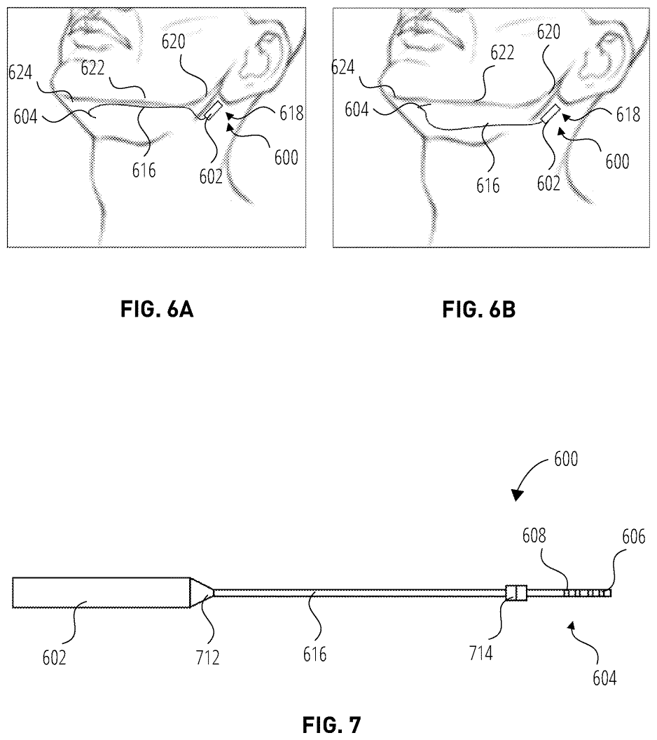

FIG. 6A to FIG. 7 illustrate an alternative neurostimulator implant 600 and implantation site in the subject. As illustrated, the neurostimulator implant 600 has a housing portion 602 and an electrode lead 604 extending from the housing portion 602. The electrode lead 604 includes electrodes 606, 608. The housing portion 602 and the electrode lead 604 are the same as described above with reference to FIG. 1 and FIG. 2.

In this example, the neurostimulator implant 600 also includes a connecting lead 616 extending between the housing portion 602 and the electrode lead 604. The connecting lead 616 may be an integral part of the electrode lead 604. That is, the connecting lead 616 may be a part of the electrode lead 604, located between the housing portion 602 and the electrodes 606, 608. Alternatively, as illustrated, the connecting lead 616 may be connected to the housing portion 602 by a lead-housing connector 712. Additionally, or alternatively, the connecting lead 616 may be connected to the electrode lead 604 by a lead-lead connector 714. The lead-housing connector 712 and/or the lead-lead connector 714 may be removable connectors, or permanent connectors that are connected during implantation. In this way, the neurostimulator implant 600 may be implanted as one piece, two pieces, or three pieces.

The connecting lead 616 increases the length of the electrode lead 604 so that the electrode lead 604 is longer than the example described above with reference to FIG. 1, FIG. 2 and FIG. 3B. In particular, the connecting lead 616 increases the distance between the housing portion 602 and the electrodes 606, 608.

As shown in FIG. 6A and FIG. 6B, this example neurostimulator implant 600 is implanted such that the housing portion 602 is implanted in a subcutaneous pocket 618 formed posteriorly of the mandibular ramus 620 of the subject. The subcutaneous pocket 618 is formed behind the mandibular ramus 620, below the ear, and anteriorly of the levator scapulae muscle at the side of the neck. In examples, the pocket 618 is defined between two muscle layers. The subcutaneous pocket 618 may include any space between the skin and the platysma muscle, or any intermuscular tissue between the platysma and muscles of the mandibular ramus 620.

From the housing portion 602, the connecting lead 616 extends towards the posterior edge of the mandibular symphysis 624 of the subject. In the example of FIG. 6A the connecting lead 616 extends along the jawline 622 of the subject. In the example of FIG. 6B the connecting lead 616 extends transversely across the submandibular tissue. In both examples, the electrode lead 604 is implanted with the electrodes 606, 608 in the positions illustrated in FIG. 3B—e.g., with at least some of the electrodes 606 being proximate to the distal branch of the hypoglossal nerve. In this example, the electrodes 606 may only target the distal branch of the hypoglossal nerve, and other electrodes 608 may optionally be provided to target the geniohyoid muscle. In other examples, the electrode lead may be positioned to stimulate plural branches of the hypoglossal nerve.

The housing portion 602 can be implanted through an incision. The connecting lead 616 (in some examples the electrode lead 604 as well) can be implanted by tunnelling. The tunnelling may extend from the incision made for the housing portion 602. The housing portion 602 may be connected to the connecting lead 616 (e.g., by lead-housing connector 712) after implanting the connecting lead 616 and during implantation of the housing portion 602.

During implantation of the connecting lead 616, the connecting lead 616 (which may have the electrode lead 604 attached or integrated) may be implanted from the subcutaneous pocket 618, along the illustrated implantation route, and may emerge supracutaneously from the skin close to the posterior edge of the mandibular symphysis 624. An incision may be made for emergence of the connecting lead 616. From this position the electrode lead 604 can be connected (e.g., via the lead-lead connector 714), or the electrode lead 604 may already be attached to the connecting lead 616 (e.g., integrally formed with the connecting lead 616). The electrode lead 604 can then be implanted into the position for neural and/or muscle stimulation as described above with reference to FIG. 3A and FIG. 3B. A delivery device, as described above, may be used to implant the electrode lead 604. The electrode lead 604 may be implanted through the incision from which the connecting lead 616 emerges close to the posterior edge of the mandibular symphysis 624.

In this example, an external device for providing power and/or communications to the neurostimulator implant 600 may be located on the neck of the subject. The external device may be integrated into a collar or scarf, for example. The external device may provide wireless power transfer and/or wireless communications as described above.

In all of the above examples the stimulation controller 202 is configured to generate stimulation signals at the electrodes 106, 108, 606, 608 in order to provide neural and/or muscle stimulation. The stimulation controller 202 is configured to individually control the electrodes 106, 108, 606, 608 so as to form monopolar, bipolar, or tripolar electrode configurations for stimulation. In this way, the electrodes 106, 108, 606, 608 can be tested post-implantation to determine which are best positioned to stimulate the hypoglossal nerve 306 and/or geniohyoid muscle 310 to treat sleep apnea. The electrodes 106, 108, 606, 608 can be tested and an assessment of their stimulation effect can be made based on movement of the subject's muscles, including by observing opening of the airway and/or protrusion of the tongue. The electrodes 106, 108, 606, 608 can be tested in different monopolar, bipolar and tripolar configurations to determine which to use for treatment.

In addition, the electrodes 106, 108, 606, 608 can be controlled as a first set of electrodes 106, 606 and a second set of electrodes 108, 608. The first set of electrodes 106, 606 can be controlled to provide stimulation of the distal branch 308 of the hypoglossal nerve 306, and the second set of electrodes 108, 608 can be controlled to provide direct muscle stimulation of the geniohyoid muscle 310.

In some examples, the electrodes 106, 108, 606, 608 may be controlled to provide neural stimulation of the distal branch 308 of the hypoglossal nerve 306. In some examples, the electrodes 106, 108, 606, 608 may be controlled to provide neural stimulation of plural branches of the hypoglossal nerve 306.

In some examples, the electrodes 106, 108, 606, 608 may be controlled to provide direct muscle stimulation of the geniohyoid muscle 310.

In some examples, the electrodes 106, 108, 606, 608 may be controlled to provide both neural stimulation of the distal branch 308 of the hypoglossal nerve 306 and direct muscle stimulation of the geniohyoid muscle 310.

The electrodes 106, 108, 606, 608 can be controlled to deliver different stimulation signals. That is, the electrodes 106, 108, 606, 608 can be controlled to provide neural and/or muscle stimulation with different stimulation characteristics.

For example, a first bipolar pair of electrodes may be controlled to deliver a first stimulation pulse having a first amplitude and pulse width, and a second bipolar pair of electrodes may be controlled to deliver a second stimulation pulse having a second amplitude and pulse width. The pulse width may also be referred to as the duration of the pulse, while the amplitude of the pulse may also be referred to as the intensity of the pulse. Both bipolar pairs may be arranged to stimulate the distal branch 308 of the hypoglossal nerve 306, with the differences in the stimulation pulses accounting for differences in the positions of the electrodes relative to the distal branch 308 of the hypoglossal nerve 306.

In another example, a first bipolar pair of electrodes may be controlled to deliver a first stimulation pulse to the distal branch 308 of the hypoglossal nerve 306, and a second bipolar pair may be controlled to deliver a second stimulation pulse directly to the geniohyoid muscle 310. The stimulation pulse delivered to the geniohyoid muscle 310 may have a larger amplitude than the stimulation pulse delivered to the distal branch 308 of the hypoglossal nerve 306, as direct muscle stimulation requires higher power. Similarly, a stimulation pulse delivered to the geniohyoid muscle 310 may have a longer pulse width than the stimulation pulse delivered to the distal branch 308 of the hypoglossal nerve 306, as direct muscle stimulation requires more time for de-polarisation between pulses.

Stimulation of the distal branch 308 of the hypoglossal nerve 306 and stimulation of the geniohyoid muscle 310 may be provided simultaneously or sequentially (non-simultaneously). Advantageously, sequential stimulation of the distal branch 308 of the hypoglossal nerve 306 and stimulation of the geniohyoid muscle 310 may reduce the maximum power usage at any one point, thereby allowing for lower wireless power transfer and/or a smaller battery, if provided.

The stimulation controller 202 may generate pulse trains for stimulating the distal branch 308 of the hypoglossal nerve 306 and/or the geniohyoid muscle 310 and optionally other branches of the hypoglossal nerve 306. The pulse trains may be different for different bipolar or tripolar electrode configurations.

As described above, the neurostimulator implant 100, 600 may include a sensor 212 for sensing one or more physiological parameter, including one or more of: a respiration rate of the subject; an electromyograph, EMG, of the subject, an electrocardiogram, ECG, of the subject, a blood oxygen saturation of the subject, a body temperature of the subject, a heart rate of the subject, and a blood pressure of the subject.

In other examples, the at least one physiological parameter may include a movement of the subject, for example at least one of: a respiratory movement of the subject, a mandibular movement of the subject, a thoracic movement of the subject, and a diaphragm movement of the subject. Such movement may be detected by the sensor 212 of the neurostimulator implant 100, 600, or by a sensor on the external device (wearable), or by a separate sensor in communication with the neurostimulator implant 100, 600.

In other examples, the sensor 212 on the neurostimulator implant 100, 600, and/or an external sensor, may detect a physiological parameter or movement of the subject indicative of sleep apnea, for example at least one of: a lapse is respiratory rate, an electromyograph, EMG, profile indicative of sleep apnea, an electrocardiogram, ECG, profile indicative of sleep apnea, a decrease in blood oxygen saturation, an increase in body temperature, an increase in heart rate, an increase in blood pressure, or a respiratory, mandibular, thoracic or diaphragm movement. The stimulation controller 202 may determine the onset of sleep apnea based on the sensed physiological parameter or movement.

The stimulation controller 202 may then generate a stimulation signal in response to the detected at least one physiological parameter or movement of the subject, or based on a determination of the onset of sleep apnea.

In examples, the stimulation controller 202 may adapt the parameters of the stimulation signal based on the detected physiological parameter, movement of the subject, or determination of the onset of sleep apnea. In particular, the stimulation signal may be adapted in terms of period/frequency and/or amplitude and/or pulse width and/or pulse interval. The adaptation of the stimulation signal may be different for neural and muscle stimulation.

In this way, the stimulation controller 202 and sensor 212 provide closed loop control of neural and/or muscle stimulation, so that the stimulation is provided when the physiological parameter and/or movement of the subject indicate that treatment would be beneficial, or when the onset of sleep apnea is detected.

In some examples, the stimulation signal generated by the stimulation controller 202 may be timed to the detected physiological parameter or movement of the subject. For example, the stimulation controller 202 may provide neural and/or muscle stimulation in pulses or pulse trains simultaneously with inspiration of the subject, in order to open the subject's airway and/or protrude the tongue during inspiration. The stimulation may be initiated at the start or predicted start of inspiration, and may be applied for a time corresponding to the inspiration.

In other examples, particularly if the neurostimulator implant 100, 600 does not comprise a sensor, the stimulation controller 202 may be configured to generate the stimulation signal to provide neural and/or muscle stimulation in an open loop configuration. In this example, the stimulation may be provided at regular intervals corresponding to an average breathing rate. The average breathing rate may be a subject-specific average breathing rate determined by observation of the subject before implantation of the neurostimulator implant 100, 600. In other examples, the average breathing rate may be a multi-subject average breathing rate, for example based on determined or known averages for subjects with similar physiology and/or Apnea-Hypopnea Index (AHI) ratings.

The stimulation controller 202 is configured to generate a stimulation signal during a stimulation time period and is configured not to generate a stimulation signal during a non-stimulation time period. The stimulation time period may also be referred to as the stimulation-on time period or stim-on. The non-stimulation time period may also be referred to as stimulation-off time period or stim-off. As described above, the stimulation time period may be synchronised with a detected physiological parameter or movement. Each stimulation time period may include a single stimulation pulse, multiple stimulation pulses, or a stimulation pulse train. Advantageously, a pulse train may provide improved stimulation with lower power consumption. Within the stimulation time period, the stimulation signal may operate according to a duty cycle, where the stimulation signal can be set to be on for a given interval and then be off for another interval and repeating those intervals. For example, the stimulation signal can be set by the user to have a duty cycle sequence in which there is a first interval, where the stimulation signal may be on for 5 seconds, and then a second interval, where the stimulation signal is off for 3 seconds. As the stimulation signal may be on for 62.5% of the time, this duty cycle may be referred to as a 62.5% duty cycle. Within the duty cycle on interval, or when the neurostimulator 100 is not operating according to a duty cycle, the stimulation signal may be may be referred to as providing “continuous” stimulation. However, it will be understood that this does not indicate a single continuous stimulation, but a series of pulses, which may be in the form of pulse trains, as will be discussed below. The neurostimulator 100 may operate according to a duty cycle or not regardless of the type of stimulation provided by the stimulation signal.

### Pulse Stimulation Characteristics and Parameters

The below description describes different pulses and pulse stimulation that may be provided by the neurostimulator implant 100, 600 to the distal branch 308 of the hypoglossal nerve 306.

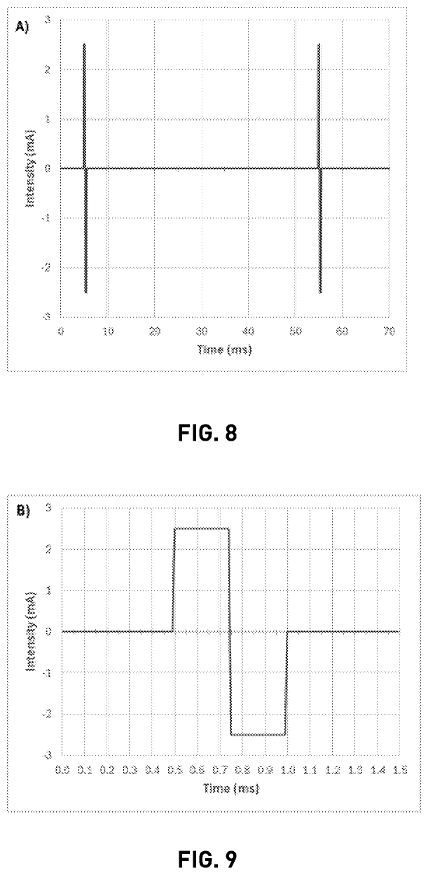

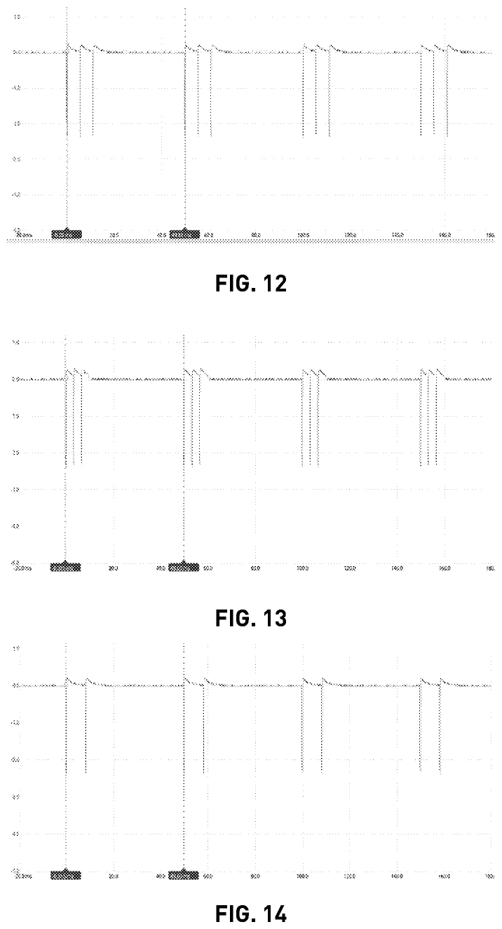

FIG. 8 to FIG. 11 show different characteristics of pulses that may be included in the stimulation signal provided by the neurostimulator implant 100, 600. The stimulation signal may comprise one or more pulse stimulations. The stimulation signal may comprise a single pulse, a series of pulses, a pulse train stimulation, and so on. FIG. 12 to FIG. 15 show different characteristics of pulse train stimulations.

FIG. 8 shows an example stimulation signal that may be provided by the neurostimulator implant 100, 600 to the distal branch 308 of the hypoglossal nerve 306 as described above. FIG. 8 shows an example of a multiple pulse stimulation signal, comprising a first biphasic pulse with an intensity of +/−2.5 mA at 5 ms and a second biphasic pulse at 55 ms, such that the stimulation signal has a frequency of 20 Hz. While only two pulses are illustrated, it will be understood that these pulses may be part of a longer series of pulses.

With the example pulse stimulation described in relation to FIG. 8, the hypoglossal nerve 306 is stimulated using a series of charge-balanced biphasic pulsed electrical signals. The first phase of the pulse may be either anodic or cathodic. The selection of anodic or cathodic first phase may be selected by a user, or may be pre-programmed into the stimulation controller 202. The second phase will be automatically selected to be the opposite polarity of the first phase. For instance, if the first phase is cathodic, then the second phase is anodic, and if the first phase is anodic, then the second phase is cathodic. Balance of the charge implies that the amount of charge delivered by the first phase pulse is matched by the amount of charge withdrawn by the second phase pulse.

In some examples, charge balancing of each pulse is achieved using an active mode, in which the amplitude and width of the second phase of each pulse is the same as for the first phase of each pulse. FIG. 9 shows an example of a single biphasic pulse (as shown in FIG. 8) on a smaller time scale. As illustrated in FIG. 9, the single biphasic pulse has an active charge balance, with the amplitude and width of the first phase (anodic phase) being the same as the amplitude and width of the second phase (cathodic phase). In this example the pulse width is approximately 250 microseconds, and the amplitude is 2.5 mA, as in FIG. 8.

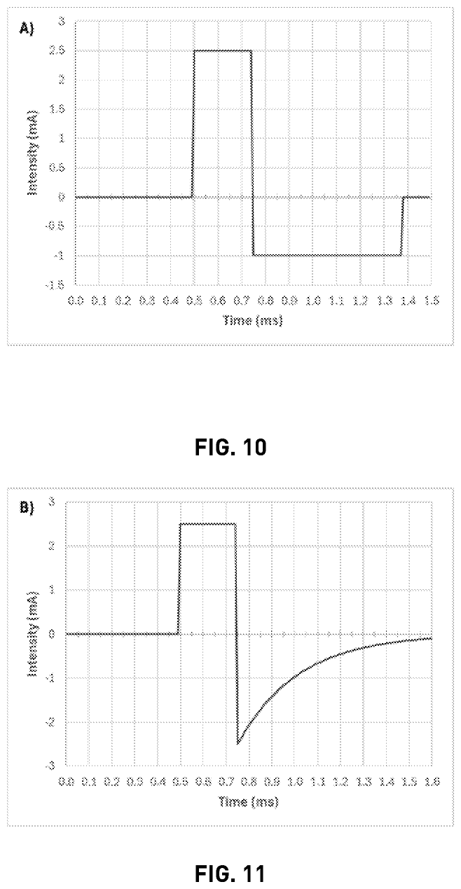

In other examples, charge-balancing of each pulse is achieved in a passive mode. In these examples, the width of the second phase of each pulse can be longer or shorter than the width of the first phase, as shown in FIG. 10. FIG. 10 shows a single pulse with passive charge balancing, in which the second phase (cathodic phase) has a lower amplitude and longer width than the first phase (anodic phase).

If the width of the second (cathodic) phase is longer, then the amplitude of the second (cathodic) phase is adjusted to be smaller, such that the charge of the second phase is equal to the charge of the first phase. Conversely, if the width of the second (cathodic) phase is shorter, then the amplitude is adjusted to be larger, such that the charge of the second phase is equal to the charge of the first phase. It will be appreciated that, as illustrated in FIG. 10, the area under the curves of the first and second phases represents the charge, and thus these areas are equal.

FIG. 11 shows another example of passive charge-balancing. FIG. 11 shows a pulse illustrating passive charge balance using capacitive coupling. In such an example, charge balancing is achieved by introducing a coupling capacitor between the stimulation controller 202 and the electrodes 106, 108. The coupling capacitor may be connected in series between the stimulation controller 202 and the electrodes 106, 108. When the first phase of stimulation is delivered, the series connection of the electrodes and coupling capacitor is short-circuited and thus the capacitor discharges to the tissue with inverse polarity and for a period such that the delivered capacitor charge matches the charge delivered by the first phase.

The width and intensity of the first phase (anodic phase) are 250 microseconds and 2.5 mA respectively. In the example in FIG. 11, the second phase (cathodic phase) is coupled capacitively so the intensity of the second phase can be set to exponentially decay toward zero within a time such that the charge of the second phase is the same as that of the first phase.

The amplitude of the first phase of the stimulating signal may be between 0 mA and 20 mA. Preferably, the amplitude of the first phase of the stimulating signal may be between 2 mA and 10 mA, for example about 5 mA.

The width of the first phase of the stimulating signal can be set to any value between 1 and 1000 microseconds.

It will be appreciated that combinations of amplitude and width must account for safe stimulation in terms of the surface charge delivered in a given area of neural tissue.

Single stimulation pulses can be delivered to the tissue at a rate set to a value between 1 and 100 Hz.

The pulse stimulations described with reference to FIG. 8 to FIG. 11 may be applied as single pulses, or as a series of pulses constituting a multi-pulse stimulation. For example, during a stimulation time period in which stimulation is applied, one or more pulses may be generated.

In other examples, stimulation pulses may be generated as a pulse train. FIG. 12 to FIG. 15 show different examples of pulse train stimulations.

In a pulse train stimulation mode, the hypoglossal nerve 306 will be stimulated using a train of two or more charge-balanced biphasic pulses. Each of the pulses in the train has characteristics similar to those described above for a single pulse stimulation.

FIG. 12 shows an example of a stimulation signal comprising pulse train stimulation. FIG. 12 shows an oscilloscope generated trace of pulse train stimulation consisting of three pulses per pulse train. Four pulse trains are shown, but it will be understood that a stimulation signal providing pulse strain stimulation may comprise a larger number of pulse trains. In this example, the stimulation signal comprises pulse trains that are generated at a rate of 20 Hz, such that a new pulse train is generated every 50 ms. The time between pulse trains may be referred to as the intertrain time, such that the frequency of the pulse trains may be referred to as the intertrain rate. The time between pulses in each train is 5.5 ms. This time may be referred to as the intratrain time, such that the frequency of the pulses within an individual pulse train may be referred to as the intratrain rate. In this example, the intratrain rate is 180 Hz. In this example, each pulse is charge-balanced using a passive mode with capacitive coupling. The X-axis is time in ms, and the Y-axis is voltage measured in the oscilloscope using a 1 kΩ resistor for a current intensity of 2.5 mA for the first phase. The pulse width of the first phase is 250 microseconds.

In this example of pulse train stimulation mode, each pulse train comprises three charge balanced pulses using capacitively coupled passive mode. Each of the three pulses is identical to each other in terms of the intensity of the first phase (2.5 mA) and pulse width (250 microseconds). Additionally, the individual pulses within the pulse train are equally spaced apart from each other on the time axis. The intratrain frequency, that is the rate of the pulses within a pulse train, may be 50 and 1,000 Hz.

FIG. 13 shows a further example of pulse train stimulation. FIG. 13 shows an oscilloscope generated trace of a portion of a stimulation signal with pulse train stimulation consisting of three pulses per train. Four pulse trains are shown. The intertrain rate is 20 Hz and the intratrain rate in each train is 300 Hz. Each pulse is charge-balanced using a passive mode with capacitive coupling. The X-axis is time in ms, and the Y-axis is voltage measured in the oscilloscope using a 1 kΩ resistor for a current intensity of 2.5 mA for the first phase. The pulse width of the first phase is 250 microseconds.

As illustrated in FIG. 13, in this example the pulse train comprises three pulses in which the intratrain rate is 300 Hz. Typically, the intertrain rate may be measured in relation to the first pulse of each train. In the example of FIG. 13, the intertrain rate is 20 Hz.

FIG. 14 shows another example of pulse train stimulation. FIG. 14 shows an oscilloscope generated trace of a portion of a stimulation signal with pulse train stimulation consisting of two pulses per pulse train. Four pulse trains are shown. The intertrain rate is 20 Hz and the intratrain rate is 120 Hz. Each pulse is charge-balanced using a passive mode with capacitive coupling. The X-axis is time in ms, and the Y-axis is voltage measured in the oscilloscope using a 1 kΩ resistor for a current intensity of 2.5 mA for the first phase. The pulse width of the first phase is 250 microseconds.

As shown in FIG. 14, each pulse train comprises two pulses. As with the previous example, each of the two pulses is identical to each other in terms of the intensity of the first phase (2.5 mA) and pulse width (250 microseconds). The intratrain times shown in FIG. 12 are substantially identical, as are the intertrain times. The intratrain frequency is 120 Hz in this example.

While the examples described herein in relation to FIG. 10, FIG. 11, and FIG. 12 have substantially constant pulse dimensions, intertrain timings/rates and intratrain timings/rates across the pulse train stimulation, it will be understood that this is not essential. Pulse trains within a series of pulse trains may differ from each other such that a first pulse train may differ from a second pulse train, which may in turn differ from a third pulse train. The pulses within the pulse train can also be set to be different from each other in terms of their intensity and width. The intratrain timings may also be varied within a pulse train. The intertrain timings may also be varied.

As discussed previously, the stimulation signal may operate according to a duty cycle. Where the neurostimulator is operating to provide pulse train stimulation signal according to a duty cycle, the pulse trains will be provided during the on interval of the duty cycle while no pulse trains will be provided during the off interval. The on and off times may be between 0.5 to 30 seconds. For example, the delivery of train stimulation can have a duty cycle sequence in which the stimulation is on for 6 seconds and is off for 4 seconds, a 60% duty cycle.

As can be understood from the description herein, a pulse train stimulation comprises a group of pulses repeated at an intertrain frequency, wherein the pulses may be grouped together within the intertrain period. In this way, pulse train stimulation comprises a plurality of train intervals, each train interval comprising the group of pulses forming the pulse train and a subsequent period of inactivity. The next train interval then begins with the next pulse train. In this way, each train interval comprises a portion thereof having group of pulses, and a portion that is pulse-free. The pulse-free portion may be of longer duration than the portion with pulses. When the neurostimulator implant 100, 600 is not operating in a pulse train mode, it may operate single or multiple pulses, where the multiple pulses would be not be grouped as with the pulse train mode. In a pulse train mode, a pulse train will become a series of single pulses if the intratrain and intertrain rates become equal. In pulse train mode, the intertrain rate is always less than the intratrain rate.

### EXAMPLES

Minimally invasive stimulation of the hypoglossal nerve was employed for the treatment of moderate to severe obstructive sleep apnea (OSA) in a first subject.

A first subject was an overweight (Body Mass Index, BMI of 28.4 kg/m²) 36 years old male subject presented to the ear, nose, and throat (ENT) investigator with a diagnosis of severe OSA as indicated by an apnea and hypopnea index (AHI) of 48.5 events per hour, disturbance of sleep as indicated by a score of 16 out of 24 in the Epworth sleepiness scale (ESS) and obstruction of the upper airway verified by a drug-induced sleep endoscopy (DISE). The subject refused to be treated using the standard continuous positive airway pressure (CPAP) therapy. After consenting to the procedure, the subject underwent the percutaneous implanting of a cylindrical (1.3 mm diameter) eight electrode array using an anterolateral approach as described herein.

Briefly, the first subject was sedated and monitored under DISE conditions. After sleep conditions were induced, ultrasound imaging of the submental region was done to verify the position of the targeted hypoglossal nerve and adjacent structures (muscles, vasculature and glands). A needle was then inserted at the most anterior medial point under the chin just behind the posterior edge of the mandible and then advanced laterally in an anterior-posterior trajectory to the neighborhood of the target nerve using ultrasound imaging guidance. The electrode array was then deployed and advanced near the target nerve. Then, the electrical stimulation was turned on while a naso-laryngeal endoscope captured video of the upper airway.

In this example, the stimulation under DISE conditions consisted of a single passively balanced biphasic pulse with a cathodic phase 300 μs wide, repeated at a rate of 10 Hz. The intensity of the signal was increased slowly until tongue motion was observed both at the upper airway and at the mouth. At this point, the pulse rate was increased to 20 Hz. After stimulation was verified, the needle was removed and the lead anchored to prevent migration. The small puncture at the introduction point was sutured and the first subject awakened and sent to recovery before being discharged home.

One day after the implant, a full CT scan of the neck and head was obtained to verify the position of the electrode array. This was followed by a stimulation programming section in which the pulse train mode was activated.

In this section, the pulse rate and intensity of the stimulation were optimized to provide comfortable stimulation that provided the desired motion (protrusion, depression) and tone of the tongue. In this example, this was provided by a 3-pulse train, at a 20 Hz intertrain rate, 180 Hz intratrain rate, 300 μs pulse width, and 2.5 mA intensity. The signal was duty-cycled to provide stimulation during most of the inhalation periods of respiration.

A polysomnography session was scheduled for the night at the 4 days post-implant to evaluate the effect of treatment, followed by an evaluation of the treatment effect on sleepiness using the ESS questionnaire. The AHI was reduced by treatment to 17.1 events per hour, which represents a reduction of 65% relative to baseline, going from severe to slightly moderate OSA (AHI below 15 events per hour is mild OSA). The Epworth scale score went down to 1, representing a reduction of 93.8% relative to baseline meaning that the first subject sleepiness sensation during the day was reduced dramatically. The first subject also felt very satisfied with the treatment.

Minimally invasive stimulation of the hypoglossal nerve was employed for the treatment of moderate to severe obstructive sleep apnea (OSA) in a second subject.

The second subject was an obese (BMI of 31.9 kg/m²), 60 years old female subject referred to the ENT investigator with a diagnosis of moderate (almost severe OSA) with an AHI of 27.4 events per hour (AHI of at least 30 events per hour indicates severe OSA). Although the subject did not score to suffer from significant sleepiness (ESS of 1 out of 24), her BMI, age and other comorbidities put her at risk if not treated. The subject refused treatment with CPAP therapy and was deemed to be a good candidate during a DISE evaluation of the upper airway. After consenting to the procedure, the subject underwent implanting of the electrode array as described in the previous example. As with the previous subject, a full CT scan of the neck and head was obtained one day after the implant to verify the position of the electrode array.

This was followed by a stimulation programming section in which the pulse train mode was activated. In this section, the pulse rate and intensity of the stimulation were optimized to provide comfortable stimulation that provided the desired motion (protrusion, depression) and tone of the tongue. In this example, this was provided by a 2-pulse train, at an intertrain rate of 20 Hz, 180 Hz intratrain rate, 300 μs pulse width, and 3.0 mA intensity. The signal was duty-cycled to provide stimulation during most of the inhalation periods of respiration. The polysomnography session at the night of the 4 days post-implant indicated that treatment had reduced the AHI to 8.0 events per hour, representing a reduction of 70.8% relative to baseline and from highly moderate OSA to slightly mild (an AHI of 5 to 15 events per hour is considered mild). The second subject felt very satisfied with the treatment.

An additional 8 subjects were evaluated, providing an example representation with a sample of 10 subjects, which provides enough statistical power (at least 80%) to test if the difference in mean AHI between pre-treatment (baseline) and treatment is significant at an alpha level of 5%. Half of the subjects in the sample were males. The mean age was 51 years old with a 43.6 to 58.2 years old 95% confidence interval (95% CI). Mean BMI was 27.4 kg/m² with a 25.1 to 29.2 kg/m² 95% CI.

Overall, the mean pretreatment AHI was 36.8 events per hour with a 23.5 to 50.1 events per hour 95% CI. The mean AHI at 4-day post implant was 11.1 events per hour, with a 3.2 to 19.0 events per hour 95% CI. A standard paired t-test indicated that the reduction in AHI due to treatment is significant (p<0.0001). The mean % reduction relative to pre-treatment was 72.7% (61.3% to 84.1% 95% CI). Additionally, all subjects experienced a reduction of at least 50% in the AHI, with 90% of them experiencing an AHI below 20 events per hour due to treatment, while 6 out of the 10 subjects had AHI consistent with mild OSA and 2 with no OSA (AHI below 5 events per hour).

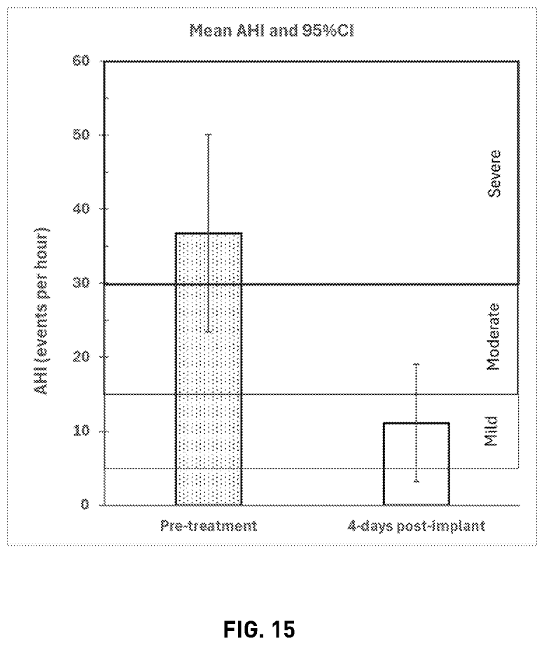

FIG. 15 shows a bar graph of the mean Apnea-Hypopnea Index (AHI) before treatment (pre-treatment) and at 4-days post-implant obtained from polysomnography of the 10 subjects described above diagnosed with moderate-to-severe apnea. The error bars are the 95% confidence interval (95% CI). The difference between the mean values is statistically significant (p<0.0001). The mean AHI pre-treatment is within the severe OSA range, while the mean post-implant AHI is within the mild OSA range.

Throughout the description and claims of this specification, the terms “pulse width” and “duration” may be understood to be interchangeable. Similarly, the terms “intensity” and “amplitude” in relation to the pulses may also be understood to be interchangeable.

Throughout the description and claims of this specification, the words “comprise” and “contain” and variations of them mean “including but not limited to”, and they are not intended to (and do not) exclude other components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A neurostimulator implant for treating sleep apnea, the neurostimulator implant comprising an electrode lead and a stimulation controller,
wherein the electrode lead comprises a plurality of electrodes spaced along the electrode lead, the electrode lead being implantable in a subject such that at least some of the electrodes are implanted to stimulate a distal branch of the hypoglossal nerve, and
wherein the stimulation controller is configured to independently control the plurality of electrodes.

2. The neurostimulator implant of claim 1, wherein the stimulation controller is configured to control the plurality of electrodes to deliver charge-balanced biphasic pulses.

3. The neurostimulator implant of claim 1 or 2, wherein the stimulation controller is configured to control the plurality of electrodes to deliver pulse train stimulation.

4. The neurostimulator implant of claim 3, wherein the stimulation controller is configured to control the plurality of electrodes to deliver different pulse train stimulation at different electrodes.

5. The neurostimulator implant of any one of claims 1 to 4, wherein the stimulation controller is configured to selectively operate the plurality of electrodes in at least one electrode array comprising a set of the plurality of electrodes.

6. The neurostimulator implant of claim 5, wherein the or each electrode array comprises a bipolar electrode configuration or a tripolar electrode configuration.

7. The neurostimulator implant of claim 5 or 6, wherein the stimulation controller is configured to selectively operate the plurality of electrodes in a first electrode array and a second electrode array.

8. The neurostimulator implant of claim 7, wherein the stimulation controller is configured to operate the first electrode array and the second electrode array with different stimulation parameters, including stimulation amplitude and frequency.

9. The neurostimulator implant of any one of claims 1 to 8, wherein the plurality of electrodes comprise a first set of electrodes and a second set of electrodes, the first set of electrodes being arranged on the electrode lead to stimulate the distal branch of the hypoglossal nerve when implanted, and the second set of electrodes being arranged on the electrode lead to stimulate a geniohyoid muscle of the subject when implanted.

10. The neurostimulator implant of claim 9, wherein the stimulation controller is configured to control the first set of electrodes and the second set of electrodes to deliver different stimulation.

11. The neurostimulator implant of claim 9 or 10, wherein the stimulation controller is configured to control the electrodes to selectively stimulate the distal branch of the hypoglossal nerve with the first set of electrodes and the geniohyoid muscle with the second set of electrodes.

12. The neurostimulator implant of any one of claims 9 to 11, wherein the stimulation controller is configured to control the first set of electrodes to define a first electrode configuration, and to control the second set of electrodes to a define a second electrode configuration, each electrode configuration comprising a bipolar electrode configuration or a tripolar electrode configuration.

13. The neurostimulator implant of any one of claims 9 to 12, wherein the stimulation controller is configured to control the electrodes to sequentially stimulate the hypoglossal nerve and the geniohyoid muscle during use.

14. The neurostimulator implant of any one of claims 9 to 12, wherein the stimulation controller is configured to control the electrodes to simultaneously stimulate the hypoglossal nerve and the geniohyoid muscle during use.

15. The neurostimulator implant of any one of claims 1 to 14, wherein the neurostimulator implant comprises a wireless power receiver for receiving wireless power from an external device, for example a wearable device.

16. The neurostimulator implant of any one of claims 1 to 15, wherein the neurostimulator implant comprises a battery.

17. The neurostimulator implant of any one of claims 1 to 16, wherein the neurostimulator implant further comprises a sensor for detecting a physiological parameter or movement of the subject.

18. The neurostimulator implant of claim 17, wherein the stimulation controller is configured to receive a sensor signal from the sensor and to control the plurality of electrodes based on the detected physiological parameter or movement.

19. The neurostimulator implant of claim 18, wherein the stimulation controller is configured to control the plurality of electrodes to dynamically provide stimulation to follow a detected breathing phase and/or breathing rate of the subject and/or to dynamically provide stimulation in response to a detected apnea event.

20. The neurostimulator implant of claim 17 or 18, wherein the physiological parameter is a breathing phase and/or breathing rate of the subject.

21. The neurostimulator implant of any one of claims 17 to 19, wherein the physiological parameter is a heart rate of the subject.

22. The neurostimulator implant of any one of claims 1 to 21, wherein the stimulation controller is configured to generate stimulation signals based on a predetermined stimulation pattern.

23. A neurostimulator system comprising the neurostimulator implant of any one of claim 1 to claim 22 and an external sensor configured to detect a physiological parameter or movement of the subject.

24. The neurostimulator system of claim 23, wherein the external sensor comprises at least one of: a mmWave radar sensor, an RF-based sensor, a magnetic sensor, or an inductive sensor, the external sensor being configured to detect movement of the subject to determine a breathing rate and/or breathing phase and/or apnea event.

25. The neurostimulator system of claim 23 or claim 24, wherein the stimulation controller is configured to receive a sensor signal from the external sensor and to control the plurality of electrodes based on the detected physiological parameter or movement or apnea event.

26. The neurostimulator implant of any one of claim 23 to claim 25, wherein the stimulation controller is configured to control the plurality of electrodes to dynamically provide stimulation to follow a detected breathing phase and/or breathing rate of the subject and/or to dynamically provide stimulation in response to a detected apnea event.

27. A method of treating sleep apnea in a subject, the method comprising:
implanting a neurostimulator implant in the subject, the neurostimulator implant comprising an electrode lead and a stimulation controller, the electrode lead having a plurality of electrodes spaced along the electrode lead, wherein at least some of the electrodes are implanted to stimulate a distal branch of the hypoglossal nerve, and
by the stimulation controller, independently operating the plurality of electrodes to stimulate the distal branch of the hypoglossal nerve of the subject.

28. The method of claim 27, comprising, by the stimulation controller, controlling the plurality of electrodes to charge-balanced biphasic pulses.

29. The method of claim 27 or 28, comprising, by the stimulation controller, controlling the plurality of electrodes to deliver pulse train stimulation.

30. The method of claim 29, comprising, by the stimulation controller, controlling the plurality of electrodes to deliver different pulse train stimulation at different electrodes.

31. The method of any one of claims 27 to 30, comprising, by the stimulation controller, selectively operating the plurality of electrodes in at least one electrode array comprising a set of the plurality of electrodes.

32. The method of claim 31, wherein the or each electrode array comprises a bipolar electrode configuration or a tripolar electrode configuration.

33. The method of claim 31 or 32, comprising, by the stimulation controller, selectively operating the plurality of electrodes in a first electrode array and a second electrode array.

34. The method of claim 33, comprising, by the stimulation controller, operating the first electrode array and the second electrode array with different stimulation parameters, including stimulation amplitude and frequency.

35. The method of any one of claims 27 to 34, comprising, by the stimulation controller, operating the plurality of electrodes to define a first set of electrodes and a second set of electrodes, the first set of electrodes being implanted to stimulate the distal branch of the hypoglossal nerve when implanted, and the second set of electrodes being implanted to stimulate a geniohyoid muscle of the subject when implanted.

36. The method of claim 35, comprising, by the stimulation controller, controlling the first set of electrodes and the second set of electrodes to deliver different stimulation to the distal branch of the hypoglossal nerve and to the geniohyoid muscle.

37. The method of claim 35 or 36, comprising, by the stimulation controller, selectively stimulating the distal branch of the hypoglossal nerve with the first set of electrodes and the geniohyoid muscle with the second set of electrodes.

38. The method of any one of claims 35 to 37, comprising, by the stimulation controller, controlling the first set of electrodes to define a first electrode array, and controlling the second set of electrodes to a define a second electrode array, each electrode array comprising a bipolar electrode configuration or a tripolar electrode configuration.

39. The method of any one of claims 35 to 38, comprising sequentially stimulating the hypoglossal nerve and the geniohyoid muscle.

40. The method of any one of claims 35 to 39, comprising simultaneously stimulating the hypoglossal nerve and the geniohyoid muscle.

41. The method of any one of claims 35 to 40, comprising receiving wireless power from an external device.

42. The method of any one of claims 35 to 41, comprising detecting, by a sensor, a physiological parameter or movement of the subject.

43. The method of claim 42, wherein the sensor is an external sensor configured to detect a movement of the subject indicative of a breathing rate and/or breathing phase and/or apnea event.

44. The method of claim 42 or claim 43, comprising, by the stimulation controller, controlling the electrodes based on the detected physiological parameter or movement.

45. A neurostimulator implant comprising:
a housing portion having a wireless power receiver,
an electrode lead comprising a plurality of electrodes, and
a connecting lead extending between the housing portion and the electrode lead,
wherein the connecting lead comprises a length such that the neurostimulator device is implantable in a subject with the housing portion disposed posterior to the ramus of the mandible of the subject with the connecting lead extending to a submandibular location, and the electrode lead being implanted proximate to a distal branch of the hypoglossal nerve.

46. The neurostimulator implant of claim 45, wherein the connecting lead has a length of between 5 cm and 20 cm.

47. The neurostimulator implant of claim 45 or 46, wherein the connecting lead is integrally formed with the electrode lead.

48. The neurostimulator implant of claim 45 or 46, comprising a connector for connecting the connecting lead to the electrode lead.

49. The neurostimulator implant of any one of claims 45 to 48, comprising a connector for connecting the connecting lead to the housing portion.

50. A method of implanting a neurostimulator implant, the neurostimulator implant comprising a housing portion having a wireless power receiver, an electrode lead comprising a plurality of electrodes, and a connecting lead extending between the housing portion and the electrode lead, the method comprising implanting:
the housing portion in a location posterior to the ramus of the mandible of the subject,
the connecting lead extending from the housing portion to a submandibular location of the subject, and
the electrode lead being implanted proximate to a distal branch of the hypoglossal nerve.

51. The method of claim 50, wherein the connecting lead is implanted along a jawline of the subject.

52. The method of claim 50, wherein the connecting lead extends across the submandibular area.

53. The method of any one of claims 50 to 52, wherein the connecting lead extends to a position proximate to the posterior edge of the mandibular symphysis, and wherein the electrode lead extends in an anterior-posterior direction to position the plurality of electrodes proximate to the distal branch of the hypoglossal nerve.

54. The method of claim 53, comprising:
implanting the connecting lead such that it protrudes from the skin of the subject adjacent to the posterior edge of the mandibular symphysis, and
subsequently implanting the electrode lead such that the plurality of electrodes are implanted proximate to the distal branch of the hypoglossal nerve.

55. The method of claim 54, wherein implanting the electrode lead comprises using an implant delivery device comprising a delivery sheath holding the electrode lead, the delivery sheath being moved percutaneously to carry the electrode lead to the position proximate to the distal branch of the hypoglossal nerve and then withdrawn.

56. The method of any one of claims 50 to 55, comprising implanting the connecting lead by tunnelling.

57. A neurostimulator implant for treating sleep apnea in a subject, the neurostimulator implant comprising:
a stimulation controller, and
an electrode lead having a plurality of electrodes,
wherein the stimulation controller is connected with the plurality of electrodes and configured to generate a stimulation signal for stimulating a distal branch of a hypoglossal nerve of the subject during use, wherein the stimulation signal comprises a pulse train comprising a plurality of charge-balanced biphasic pulses,
wherein each charge-balanced biphasic pulse comprises:
an amplitude of between 0.1 and 20 mA, and
a pulse width of between 1 and 1000 microseconds,
wherein each pulse train comprises an intratrain frequency of between 50 and 1000 Hz, and
wherein an intertrain frequency is between 1 and 50 Hz.

58. The neurostimulator implant of claim 57, wherein each charge-balanced biphasic pulse comprises an active charge-balanced biphasic pulse comprising an anodic phase and a cathodic phase, the anodic and cathodic phases having equal amplitude and pulse width.

59. The neurostimulator implant of claim 57, wherein each charge-balanced biphasic pulse comprises a passive charge-balanced biphasic pulse comprising an anodic phase and a cathodic phase, the anodic and cathodic phases having non-equal amplitude and pulse width.

60. The neurostimulator implant of any one of claims 57 to 59, wherein each charge-balanced biphasic pulse comprises an amplitude of between 0.5 and 15 mA, for example between 0.5 and 10 mA, for example between 1 and 8 mA, for example between 1 and 5 mA, for example between 1.5 and 5 mA, for example between 1.5 and 3.5 mA, for example between 1.8 and 3.2 mA, for example between 2 and 3 mA, for example about 2.5 mA.

61. The neurostimulator implant of any one of claims 57 to 60, wherein each charge-balanced biphasic pulse comprises a pulse width of between 50 and 800 microseconds, for example between 100 and 600 microseconds, for example between 100 and 500 microseconds, for example between 100 and 400 microseconds, for example between 150 and 400 microseconds, for example between 200 and 300 microseconds, for example about 250 microseconds.

62. The neurostimulator implant of any one of claims 57 to 61, wherein the intratrain frequency is between 50 Hz and 500 Hz, for example between 50 Hz and 400 Hz, for example between 100 Hz and 400 Hz, for example between 100 Hz and 350 Hz, for example between 100 Hz and 300 Hz, for example between 120 Hz and 300 Hz, for example about 120 Hz, for example about 180 Hz, for example about 300 Hz.

63. The neurostimulator implant of any one of claims 57 to 62, wherein the intertrain frequency is between 10 Hz and 50 Hz, for example between 10 Hz and 40 Hz, for example between 15 Hz and 40 Hz, for example between 20 Hz and 30 Hz, for example 10 Hz or 15 Hz, or 20 Hz, or 25 Hz or 30 Hz or 35 Hz, or 40 Hz, or 45 Hz or 50 Hz.

64. A method of treating sleep apnea in a subject, comprising:
implanting a neurostimulator implant comprising a plurality of electrodes and a stimulation controller, the electrodes being implanted to stimulate a distal branch of a hypoglossal nerve of the subject,
by the stimulation controller, generating a stimulation signal for the electrodes and stimulating the distal branch of the hypoglossal nerve based on the stimulation signal,
wherein the stimulation signal comprises a plurality of pulse trains, each pulse train comprising a plurality of charge-balanced biphasic pulses,
wherein each charge-balanced biphasic pulse comprises:
an amplitude of between 0.1 and 20 mA, and
a pulse width of between 1 and 1000 microseconds,
wherein each pulse train comprises an intratrain frequency of between 50 and 1000 Hz, and
wherein an intertrain frequency is between 1 and 50 Hz.

65. The method of claim 64, wherein each charge-balanced biphasic pulse comprises an active charge-balanced biphasic pulse comprising an anodic phase and a cathodic phase, the anodic and cathodic phases having equal amplitude and pulse width.

66. The method of claim 64, wherein each charge-balanced biphasic pulse comprises a passive charge-balanced biphasic pulse comprising an anodic phase and a cathodic phase, the anodic and cathodic phases having non-equal amplitude and pulse width.

67. The method of any one of claims 64 to 66, wherein each charge-balanced biphasic pulse comprises an amplitude of between 0.5 and 15 mA, for example between 0.5 and 10 mA, for example between 1 and 8 mA, for example between 1 and 5 mA, for example between 1.5 and 5 mA, for example between 1.5 and 3.5 mA, for example between 1.8 and 3.2 mA, for example between 2 and 3 mA, for example about 2.5 mA.

68. The method of any one of claims 64 to 67, wherein each charge-balanced biphasic pulse comprises a pulse width of between 50 and 800 microseconds, for example between 100 and 600 microseconds, for example between 100 and 500 microseconds, for example between 100 and 400 microseconds, for example between 150 and 400 microseconds, for example between 200 and 300 microseconds, for example about 250 microseconds.

69. The method of any one of claims 64 to 68, wherein the intratrain frequency is between 50 Hz and 500 Hz, for example between 50 Hz and 400 Hz, for example between 100 Hz and 400 Hz, for example between 100 Hz and 350 Hz, for example between 100 Hz and 300 Hz, for example between 120 Hz and 300 Hz, for example about 120 Hz, for example about 180 Hz, for example about 300 Hz.

70. The method of any one of claims 64 to 69, wherein the intertrain frequency is between 10 Hz and 50 Hz, for example between 10 Hz and 40 Hz, for example between 15 Hz and 40 Hz, for example between 20 Hz and 30 Hz, for example 10 Hz or 15 Hz, or 20 Hz, or 25 Hz or 30 Hz or 35 Hz, or 40 Hz, or 45 Hz or 50 Hz.

71. A method of implanting a neurostimulator implant in a subject for treating sleep apnea, the neurostimulator implant comprising a housing portion and a flexible electrode lead extending from the housing, the method comprising:
providing a carrier element holding the electrode lead,
advancing the carrier element and the electrode lead subcutaneously from a medial submandibular location in a superior direction until a tip of the carrier element is in contact with, or proximal to, the mandibular symphysis,
rotating and advancing the carrier element and the electrode lead along a posterior-lateral-superior insertion path until an electrode of the electrode lead is disposed proximal to a distal branch of the hypoglossal nerve of the subject, and
removing the carrier element from the subject to implant the electrode lead.

72. The method of claim 71, further comprising implanting the housing portion in the submandibular area of the subject.

73. The method of claim 72, wherein the housing portion is elongate and the electrode lead extends from a first end of the housing portion, and wherein the housing portion is implanted such that the first end of the housing portion is disposed medially of the other end of the housing portion.

74. The method of claim 73, wherein the housing portion is implanted in a posterior-lateral orientation.
